(19) United States
(12) Patent Application Publication
(10) Pub. No.: US 2011/0098227 A1
(43) Pub. Date: Apr. 28, 2011

(54) Soluble notch-based substrates for gamma secretase and methods and compositions for using same

(76) Inventors: Satish Kumar Sharma, Canton, MI (US); Kenneth Bruce Rank, Zionsville, IN (US)

(21) Appl. No.: 10/717,244

(22) Filed: Nov. 19, 2003

Related U.S. Application Data
(60) Provisional application No. 60/429,206, filed on Nov. 26, 2002.

(51) Int. Cl. A61K 38/02; C07K 14/00; C07H 21/04; C12N 15/63; C12P 21/00; G01N 33/53; C12N 5/10; C12N 1/21; C12N 1/19; A61P 25/28

(52) U.S. Cl. 514/17.8; 530/350; 536/23.4; 435/320.1; 435/69.7; 435/7.1; 435/325; 435/252.3; 435/254.2; 435/366; 435/367; 435/369; 435/358; 514/1.1

(57) ABSTRACT

The present invention is directed to novel soluble substrates for γ-secretase. More particularly, the invention provides a soluble fusion polypeptide with a Notch segment containing the γ-secretase-dependent cleavage sites (γ and ε) fused to a NusA protein. Methods and compositions for making and using such a fusion protein are disclosed.

### CROSS REFERENCE TO RELATED APPLICATIONS

This application claims the benefit of the following provisional application: Application Ser. No. 60/429,206 filed 26 Nov. 2002 under 35 U.S.C. 119(e)(1).

### FIELD OF THE INVENTION

The present invention is directed to novel soluble substrates for γ-secretase. More particularly, the invention provides a soluble fusion polypeptide with a Notch segment containing the γ-secretase-dependent cleavage sites (γ and ε) fused to a NusA protein. Methods and compositions for making and using such a fusion protein are disclosed.

### BACKGROUND

Alzheimer's disease (AD) causes progressive dementia with consequent formation of amyloid plaques, neurofibrillary tangles, gliosis and neuronal loss. The disease occurs in both genetic and sporadic forms whose clinical course and pathological features are quite similar. Three genes have been discovered to date which, when mutated, cause an autosomal dominant form of AD. These encode the amyloid protein precursor (APP) and two proteins, presenilin-1 (PS1) and presenilin-2 (PS2), which are structurally and functionally related. Mutations in any of the three proteins have been observed to enhance proteolytic processing of APP via an intracellular pathway that produces amyloid beta peptide (Aβ peptide, sometimes referred to as Abeta), a 40-42 amino acid peptide that is the primary component of amyloid plaque in AD (Younkin, Brain Pathol. 1(4):253-62, 1991; Haass, J. Neurosci. 11 (12):3783-93, 1991).

Dysregulation of intracellular pathways for proteolytic processing may be central to the pathophysiology of AD. In the case of plaque formation, mutations in APP, PS1 or PS2 consistently alter the proteolytic processing of APP so as to enhance formation of Aβ 1-42, a form of the Aβ peptide which seems to be particularly amyloidogenic, and thus very important in AD. APP localizes to the secretory membrane structure including the cell surface, and has a transmembrane domain near the C-terminus (FIG. 1). Examples of specific isotypes of APP which are currently known to exist in humans include the 695-amino acid polypeptide described by Kang et al. (1987), Nature 325: 733-736 which is designated as the “normal” APP; the 751 amino acid polypeptide described by Ponte et al. (1988), Nature, 331: 525-527 (1988) and Tanzi et al. (1988), Nature, 331: 528-530; and the 770 amino acid polypeptide described by Kitaguchi et. al., Nature, 331: 530-532 (1988).

The Aβ peptide is derived from a region of APP adjacent to and containing a portion of the transmembrane domain (see FIG. 1). Normally, processing of APP at the α-secretase site cleaves the midregion of the Aβ sequence adjacent to the membrane and releases the soluble, extracellular domain of APP from the cell surface. This α-secretase APP processing creates soluble APP-a, which is not thought to contribute to AD. However, pathological processing of APP at the β- and γ-secretase sites, which are located N-terminal and C-terminal to the α-secretase site, releases the Aβ peptide. The β-secretase cleavage site is located 28 residues from the plasma membrane luminal surface and the APP γ-secretase cleavage site is located in the transmembrane region. Processing at the β- and γ-secretase sites can occur in both the endoplasmic reticulum (in neurons) and in the endosomal/lysosomal pathway after re-internalization of cell surface APP (in all cells).

Thus, the enzymatic activities of the β- and γ-secretase enzymes are targets for drug discovery (Olson et al., Curr. Opin. Drug Discovery & Develop. 4:390-401, 2001). These two enzymes act in concert to cleave APP, which is cleaved initially by β-secretase to produce membrane-bound C-terminal (CT) fragment called CT-100, which in turn serves as a substrate for the membrane-associated γ-secretase. The intramembrane-cleavage of CT-100 by presenilin 1(PS1)-dependent γ-secretase results in the production of Aβ 1-40 and 1-42. In addition, there is another cleavage event (termed gamma-like or epsilon-secretase cleavage) cleaves near residue 721 of APP at approximately 2-5 residues inside the cytoplasmic membrane boundary to generate a series of stable, C-terminal APP fragments (FIG. 1).

Notch-1 belongs to the Notch family of cell surface receptors, which play a widespread role in the assignation of cell fates. In recent years, it has been postulated that APP processing is similar to the processing of the cell surface receptor Notch 1 (Wolfe et al., J. Biol. Chem. 276:5413-5416, 2001). Indeed, it has been shown that APP and Notch-1 are competitive substrates for the putative endogenous γ-secretase. Notch-1 is an integral-membrane protein that is proteolytically processed within its ectodomain upon ligand-mediated activation. Following ligand binding, Notch-1 undergoes presenilin-dependent intramembraneous γ-secretase cleavages (Okochi, EMBO J. 2 5408-5416, 2002). The first is at 1731/1732 site (this is akin to the Aβ-like γ secretase cleavage) and the second is at the 1743/1744 site (this is akin to the ε cleavage in the generation of APP and is sometimes referred to as S3-cleavage of Notch; depicted in FIG. 1 as an “ε” cleavage). It is the ε cleavage at 1743/1744 junction of Notch that occurs toward the end of the transmembrane domain to release the Notch 1 intracellular domain (NICD). The released NICD translocates to the nucleus, where it interacts with a DNA binding protein denoted CSL (this acronym stands for three separate names given to this protein in different systems: CBF1/RBP-J in mammals; Suppressor of Hairless [Su(H)] in Drosophila and Xenopus; and Lag-1 in C. Elegans). The complex formed between NICD and CSL modifies the transcription of target genes. NICD is required for signaling pathway critical in embryonic development (Schroeter 1998; Hupert 2000).

Presenilin-dependent γ-secretase activity is required for processing of the Notch receptor to NICD (De Stropper et al., Nature 398:518-522, 1999). It has been reported that, in cells, Notch 1 and APP are competitive substrates for PS1-dependent γ-secretase cleavage (Berezovska et al., J. Biol. Chem. 276:30018-30023, 2001). As such, γ-secretase inhibitors designed to inhibit the production of pathogenic Aβ also inhibit Notch signaling. This has significant implications for the potential use of γ-secretase inhibitors as drugs in the treatment of AD. The inhibition of Notch-1 processing in the adult would lead to immunodeficiency and anemia because of the important function of Notch-1 in hematopoesis. Thus, there is a need to identify compounds that specifically inhibit APP CT-100 cleavage but do not inhibit Notch cleavage. Such compounds would serve as therapeutic agents for the intervention of AD without producing deleterious effects of inhibition of NICD production.

### SUMMARY OF THE INVENTION

The present invention provides methods and compositions for identifying compounds that do not inhibit γ-secretase mediated cleavage of Notch. These methods and compositions circumvent the problem of inhibition of NICD production that attends non-specific inhibition of γ-secretase inhibition. The present invention therefore allows for the identification of therapeutic agents for the intervention of AD without producing deleterious effects of inhibition of NICD production.

A first aspect of the present invention provides a soluble fusion protein comprising recombinant Notch protein fused to the C-terminus of a NusA protein sequence. Preferably the Notch protein comprises the transmembrane domain of Notch protein. However, the Notch protein may contain more or less of the full-length Notch protein than the transmembrane domain so long as the Notch protein contains the S3 cleavage site of Notch. The Notch protein for a specific embodiment comprises the sequence of SEQ ID NO:4 and is encoded by a nucleic acid sequence of SEQ ID NO:3. The recombinant Notch protein should be one which comprises the S3 cleavage site (i.e., the ε cleavage site) of Notch. The recombinant Notch protein may be a vertebrate Notch protein or an invertebrate Notch protein. In certain embodiments, the recombinant Notch protein is derived from mouse Notch protein having the sequence of SEQ ID NO:5 [Gen Bank accession number Z11886]. More particular embodiments, contemplate the use of a recombinant Notch protein comprises amino acids 1703 through 1860 of mouse Notch protein. Any of the soluble fusion proteins of the present invention may further comprise a C-terminal His-tag and/or a C-terminal Flag-tag. Of course, all or a portion of a human Notch sequence, e.g., the sequence of SEQ ID NO:6 [Genbank accession number M73980] also could be used.

The present invention further contemplates polynucleotides comprising a nucleotide sequence that encodes a fusion proteins described herein. An exemplary polynucleotide sequence that encodes such a fusion protein is one which comprises a sequence set forth in SEQ ID NO:1. This polynucleotide sequence encodes a fusion protein of SEQ ID NO:2. Also contemplated herein is an expression vector comprising a polynucleotide of the present invention. The expression vector preferably is one in which the polynucleotide is operably linked to a promoter to promote expression of the protein encoded by the polynucleotide in a host cell.

Recombinant host cells transformed or transfected with a polynucleotide or expression vector described herein also are encompassed by the present invention. The invention contemplates a method of producing a substrate for a γ-secretase assay comprising growing such a recombinant host cell in a manner allowing expression of the fusion protein. The method may further comprise purifying the polypeptide. In such a method, the host cell may be any host cell amenable to recombinant protein production including a mammalian host cell, a bacterial host cell and a yeast host cell. In exemplary embodiments, the host cell is a Hela cell, a human embryonic kidney cell line 293 cell, a fibroblast, or a CHO cell.

Another aspect of the present invention provides a method of producing a solubilized Notch protein, the method comprising preparing a fusion protein wherein the Notch protein is fused to the C-terminus of a NusA protein. More specifically, the method comprises a recombinant production of the fusion protein, which involves preparing an expression construct comprising a nucleic acid that encodes a fusion protein comprising a Notch protein containing the amino acids of the S3 cleavage site of Notch linked at the C-terminus of a NusA protein; transforming a host cell with the expression construct under conditions that facilitate the expression of the fusion protein; and growing the transformed host cell in culture. The method further may comprise isolating the fusion protein from the transformed host in culture. In certain embodiments, the method comprises producing the fusion protein through chemical protein synthesis. In such methods, the Notch protein preferably comprises amino acids 1703 through 1860 of mouse Notch protein.

Also contemplated herein is an in vitro method of assaying for γ-secretase mediated ε cleavage (1743/1744) of Notch protein comprising contacting a first composition comprising a mammalian γ-secretase complex or biologically active fragment thereof, with a second compositions comprising a fusion protein of the invention; and measuring cleavage of the fusion protein.

The γ-secretase complex of the above method may comprise a membrane fraction purified and isolated from mammalian cells or cells transformed or transfected with expression constructs comprising nucleotide sequences that encode the γ-secretase complex. In the above method, the fusion protein is a solubilized Notch protein prepared according to the methods discussed herein.

The invention specifically contemplates compositions comprising γ-secretase modulators identified through the screening methods described herein. Also encompassed by the present invention is a method of modulating γ-secretase activity in vivo comprising a step of administering a modulator identified by the screening methods described herein that is a γ-secretase modulator that is selective for inhibiting γ-secretase-mediated cleavage of APP as compared to γ-secretase-mediated cleavage of Notch protein to a mammal in an amount effective to modulate γ-secretase activity in cells of the mammal.

The present invention also is directed to pharmaceutical compositions comprising one or more modulators identified by the present invention and a pharmaceutically acceptable carrier. Also contemplated is a method of treating a disease or condition characterized by an abnormal γ-secretase activity comprising administering to a subject in need of treatment a pharmaceutical composition of the invention. Use of the modulators identified herein in the manufacture of a medicament for the treatment of Alzheimer's Disease is particularly contemplated.

Other features and advantages of the present invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1. Comparison of γ-secretase-like cleavage sites. The sequence surrounding the transmembrane domains of APP (SEQ ID NO:7), Notch-1 (SEQ ID NO:9), and E-cathedrin (SEQ ID NO:8) are shown. Also shown are the γ-secretase cleavage sites in APP to produce Aβ 1-40 and 1-42 as well as the ε-cleavage (ε) or γ-secretase-like cleavage sites for the three substrates.

FIG. 2. Amino acid sequence surrounding the γ-secretase-like cleavage site in Notch-1. This sequence (1703-1860; SEQ ID NO:10) was chosen for expression in E. coli. The transmembrane domain is indicated by underlining and the 1743/1744 cleavage site is indicated in bold and underlined characters. Cleavage of this Notch sequence would result in a NICD fragment containing amino acids 1744-1860.

FIG. 3. a) Constructs cloned for expression of Notch in E. coli. Four constructs are shown, all containing the amino acid sequence 1703-1860 of Notch-1. No expression was seen when either a caspase leader sequence, Ubiquitin, or the N-terminal domain of tau was used as an N-terminal tag to help drive expression. When Notch (1703-1860) was fused to the NusA protein, soluble expression in E. coli was seen. b) A schematic of the Notch F construct cloned into pET 43.1a. This construct also contains C-terminal Flag and 8His tags.

FIG. 4. IMAC isolation of NusA-Notch fusion. A schematic of the fusion protein is shown at the top. Details of expression and purification of NusA-Notch can be found in the Materials and Methods. The arrow indicates the fusion protein expression band on the 10% SDS-PAGE gel. Lane 1, crude E. coli lysate; lane 2, E. coli supernatant, lane 3, IMAC flow through; lane 4, 50 mM imidazole wash; lanes 5-9, 300 mM imidazole elution fractions; lane 10, BenchMark molecular weight marker.

FIG. 5. Western blot showing the cleavage of NusA-Notch fusion using solubilized secretase. The details of the experiment are in Example 1. Briefly, NusA-Notch fusion was incubated overnight with solubilized γ-secretase in the presence and absence of varying concentrations of DAPT (PHA-568638). The samples were electrophoresed, blotted and probed with Val1744 antibody, specific for cleavage at Val 1744.

FIG. 6. Detection of specific notch cleavage by ELISA. A schematic of the sandwich ELISA used to detect cleavage of the NusA-Notch fusion by solubilized γ-secretase is shown. Specific cleavage is detected using the Val1744 antibody.

FIG. 7. Cleavage of NusA-Notch fusion protein by γ-secretase as detected by ELISA. NusA-Notch (0.9 μM) substrate was incubated in the absence or presence of 68 μg/ml solubilized γ-secretase (enzyme) and the ELISA done according to the Materials and Methods. The data represents the average of six experiments.

FIG. 8. Characterization of notch cleavage by ELISA. The ELISA was performed as described in the Materials and Methods. When varying the Notch substrate concentration, 0.11 to 3.6 μM of NusA-Notch was incubated with 68 μg/ml solubilized γ-secretase and the data plotted using an Michaelis-Menton curve fit. When the enzyme concentration was varied, 2.1 to 68 μg/ml solubilized γ-secretase was incubated with 0.9 μM Notch substrate. The data represent the average of three experiments and were plotted using GraFit 4.0. There was an approx. 5-fold signal:background (background using enzyme alone is negligible).

FIG. 9. Inhibition of notch cleavage by compounds known to inhibit the in vitro cleavage of CT-100 by γ-secretase. The inhibition profiles for DAPT (PHA-568638) and fenchylamine (PHA-512088), as measured using the notch cleavage ELISA are shown. Also shown are the relative IC₅₀ values.

### DETAILED DESCRIPTION OF THE PREFERRED EMBODIMENTS

AD is a leading age related disorder associated with progressive dementia and pathology characterized by cortical atrophy and deposition of senile plaques and neurofibrillary tangles. A primary component of the plaques is the 40-42 amino acid long peptide, Aβ, derived from a region of APP adjacent to and containing a portion of the transmembrane domain of the full length APP. This pathogenic peptide is generated as a result of sequential processing due to β- and γ-secretases activities. Thus, the enzymatic activities of these two secretase enzymes are targets for drug discovery (Olson et al., Curr. Opin. Drug Discovery & Develop. 4:390-401, 2001).

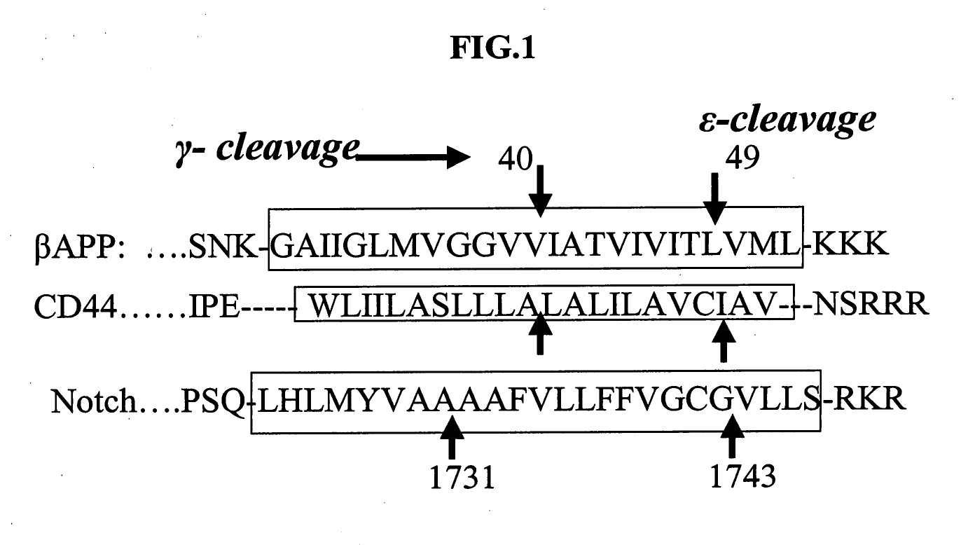

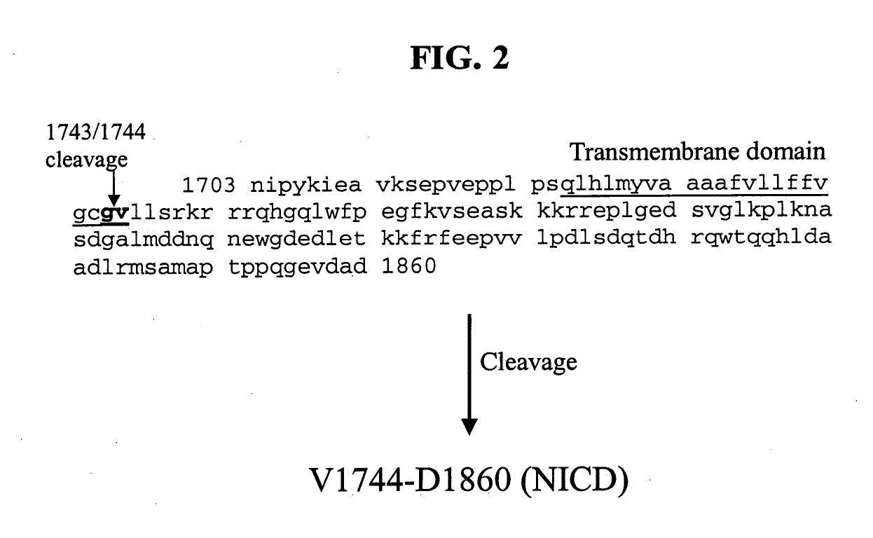

The processing of the cell surface receptor Notch has been shown to be similar to APP processing (Wolf et al., J. Biol. Chem. 276:5413-5416, 2001). FIG. 1 shows a comparison of γ-secretase-like cleavage sites in APP and Notch. As shown, the cleavage site in APP is in the middle of the transmembrane domain, while Notch is cleaved very close to the C-terminal end of its transmembrane domain. A similar ε-cleavage site has been recently reported (Marambaud et al., EMBO J., 21:1948-1956, 2002) in cathedrin E (FIG. 1). FIG. 2 shows the amino acid sequence from 1703-1860 of Notch protein including the S3 cleavage site in Notch (Steiner et al., J. Mol. Neurosci. 17:193-198, 2001). The specific cleavage at the 1743/1744 junction would produce the V1744-D1860 Notch intracellular domain (NICD), a fragment of the NICD observed in cells transfected with mΔE Notch constructs (1704 to 2183) lacking the S1 and S2 cleavage sites (Kopan et al., Proc. Natl. Acad. Sci. 93:1683-1688, 1996). Although a Notch construct (Val 1711-Glu 1809) has been reported (Esler et al Proc. Natl. Acad. Sci. 99, 2720-2725,2002), the specific cleavage by γ-secretase at the 1743/1744 junction in vitro has not been described. The specific cleavage at the 1743/1744 junction can be readily determined through the use of Val-1744 antibody (Cell Signaling Technology). This antibody is specific for cleaved Notch and does not cross react with uncleaved Notch protein.

Thus, in cells, in addition to cleaving the CT-100 fragment produced by the action of the β-secretase, PS1-dependent γ-secretase also cleaves at the ε site 1743/1744 to produce NICD. This cleaved NICD translocates to the nucleus and is involved in signaling. Therapeutic inhibition of γ-secretase activity designed to alleviate AD, also results in an inhibition of NICD production. The present invention for the first time provides methods and compositions for identifying therapeutic agents which do not inhibit the production of NICD from Notch-1.

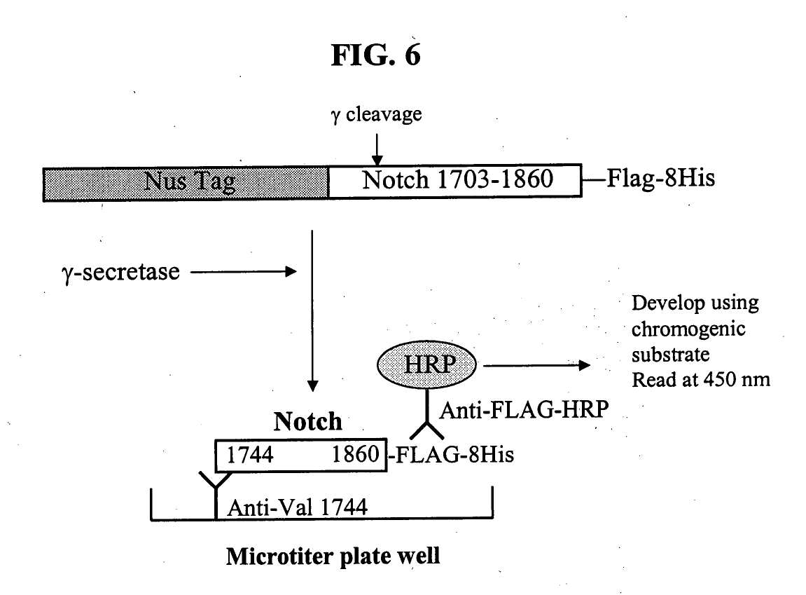

The methods of the present invention provide in vitro assays for Notch cleavage and the use of such assays in the secondary evaluation of γ-secretase inhibitors. These assays employ a soluble Notch substrate for γ-secretase, which comprises a soluble fusion protein comprising recombinant Notch protein fused to the C-terminus of a NusA protein sequence. The assays of the present invention may be performed as direct in vitro ELISA assays and/or Western blots for Notch protein cleavage by γ-secretase. The methods of the present invention provide for the production and purification of a soluble recombinant Notch protein (Asn 1703-Asp 1860) substrate expressed in a suitable cell lines, e.g., E. coli, as a fusion protein with NusA engineered to a Notch protein sequence (see FIG. 6).

Using the purified fusion protein substrate of the present invention, the inventors demonstrated specific cleavage at the 1743/1744 site in Notch using solubilized γ-secretase from HeLa cells. As described in the detailed Examples, the cleaved Notch protein can be detected using an antibody specific for Val-1744. The inventors validated that assay and showed that cleavage of the Notch protein was inhibited in a dose-dependent manner by DAPT, a well-known potent inhibitor of γ-secretase. An ELISA was developed based on anti-Val-1744 antibodies. Further validation of the in vitro Notch assay was provided by inhibition of the cleavage by γ-secretase inhibitors. Operationally, an inhibitor or a modulator is defined as compound which lowers Aβ through γ-secretase.

### I. NOTCH SUBSTRATE FOR IN VITRO NOTCH ASSAY

The Notch substrate for use in the assays of the present invention is a soluble fusion protein of a Notch polypeptide to a Nus protein. The fusion protein may be labeled or otherwise modified to facilitate the purification of the peptide, detection of the Notch fusion protein itself, or a detection of the cleavage product of the Notch fusion protein upon the action of the γ-secretase. Production of the fusion protein and exemplary modifications are described in further detail herein below.

In a preferred aspect of the present invention, the Notch substrate containing amino acids N1703-D1860 of mouse notch-1 protein (DNA Sequence accession number Z11886; see FIG. 2) joined at the N-terminal to the C-terminus of NusA. It is contemplated that the fusion polypeptide may be produced by recombinant protein production or indeed by automated peptide synthesis as discussed elsewhere in the specification. The transmembrane domain in FIG. 2 spans from amino acid 1723 through to 1744 (see FIG. 1). γ-secretase cleaves Notch at the ε-cleavage between amino acids 1743 and 1744. This is cleavage also is termed the S3-cleavage site and generates NICD (i.e., a peptide spanning amino acids V1744-D1860).

In addition to this novel fusion protein, the present invention further contemplates the generation terminal additions, also called fusion proteins or fusion polypeptides, of the Notch/NusA fusion protein substrate described above or identified according to the present invention. Moreover, while the preferred embodiments of the present invention show a Notch/NusA fusion peptide comprising N1703-D1860 of mouse Notch-1, it should be understood that the Notch protein may be derived from any source. Such a source may be mammalian, or non-mammalian. Thus, while it is preferred that the Notch-1 is derived from human, mouse, rat or another mammalian source, it is contemplated that in certain embodiments, the Notch-1 may be derived from e.g., C. elegans, Xenopus, drosophila, and other invertebrate sources.

Furthermore, it is contemplated that any Notch-1 derivative that contains the γ-secretase cleavage sites 1731/1732 (γ site) and 1743/1744 site (ε cleavage) will be useful in the fusion protein substrate of the present invention. In Notch-1, the γ-secretase cleavage site that releases NICD is located 1743/1744 site in Notch. It is contemplated that the distance between the cleavage site and the start of a NusA is about 500 amino acids in order to mimic the steric properties of the Notch γ-secretase cleavage domain. This distance may be generated from the NusA protein or it may be created by means of a heterologous peptide linker. Preferably, this region is from the NusA protein. The NusA protein domain component of the Notch/NusA fusion polypeptide may be essentially any portion of the NusA protein that allows the Notch/NusA fusion to remain soluble and therefore amenable to an in vitro assay.

NusA has previously been used to produce the soluble expression of proteins in E. coli. (See e.g., Wilkinson, et al., Bio/Technology 9, 443-448, 1991; Davis et al., Biotechnol Bioeng., 65(4):382-8, 1999). Herein, a NusA protein comprising the sequence of SEQ ID NO:14 (encoded by a nucleic acid sequence of SEQ ID NO:13) is fused to Notch. However, those of skill in the art may employ a NusA sequence other than the sequence depicted in SEQ ID NO:14 and still produce a soluble Notch/NusA fusion protein of the present invention. For example, one of skill may use a NusA protein comprising 80%, 85%, 90%, 95%, 96%, 97%, 98% or more sequence homology with the sequence of SEQ ID NO:14. Alternatively, those of skill may employ a smaller contiguous fragment NusA derived from SEQ ID NO:14, for example the fragment may be 50, 100, 150, 200, 250, 300, 350, 400, 425, 450, 475, 500, 510, 520, 530, 540, 550 or more contiguous amino acids of SEQ ID NO:14.

General principles for designing and making fusion proteins are well known to those of skill in the art. For example, fusions typically employ leader sequences from other species to permit the recombinant expression of a protein or peptide in a heterologous host. Another useful fusion includes the addition of an immunologically active domain, such as an antibody epitope, to facilitate purification of the fusion polypeptide. Inclusion of a cleavage site at or near the fusion junction will facilitate removal of the extraneous polypeptide after purification. The recombinant production of these fusions is described in further detail elsewhere in the specification. Other useful fusions include linking of functional domains, such as active sites from enzymes, glycosylation domains, cellular targeting signals or transmembrane regions.

More particularly, the present invention contemplates a fusion polypeptide in which there is a first component comprising the Notch protein containing the 1731/1732 (γ cleavage) and 1743/1744 (ε cleavage) cleavage sites attached to a second component comprising all or a portion of, the NusA protein. In additional embodiments, the fusion polypeptide further may comprise a third component which comprises a reporter gene product. In still further embodiments, the fusion polypeptides may further comprise a tagged sequence component. A particular fusion polypeptide that is contemplated is one which comprises a reporter gene product on one side of a NusA portion, a stretch of sequence containing the Notch protein with the γ-secretase cleavage sites, and tagged sequence on the other side of the Notch protein. The reporter gene product used in the fusion polypeptides of the present invention may be any reporter protein commonly used by those of skill in the art. Exemplary reporter proteins include but are not limited to luciferase; secreted alkaline phosphatase (SEAP); β galactosidase; β-glucoronidase; green fluorescent protein and chloramphenical acetyl transferase.

Other particular embodiments further contemplate a tagged sequence as a fourth component of the fusion polypeptides of the present invention. There are various commercially available fusion protein expression systems that may be used to provide a tagged sequence in this context of the present invention. Particularly useful systems include but are not limited to the glutathione S-transferase (GST) system (Pharmacia, Piscataway, N.J.), the maltose binding protein system (NEB, Beverley, Mass.), the FLAG system (IBI, New Haven, Conn.), and the 6xHis system (Qiagen, Chatsworth, Calif.). These systems are capable of producing recombinant polypeptides bearing only a small number of additional amino acids, which are unlikely to affect the biologically relevant activity of the recombinant fusion protein. For example, both the FLAG system and the 6xHis system add only short sequences, both of which are known to be poorly antigenic and which do not adversely affect folding of the polypeptide to its native conformation. Another N terminal fusion that is contemplated to be useful is the fusion of a Met Lys dipeptide at the N terminal region of the protein or peptides. Such a fusion may produce beneficial increases in protein expression and/or activity. Specific tagged sequences that are contemplated for use in the present invention include the C-terminal FLAG tag sequence DYKDDDDK (SEQ ID NO:11). In addition, the tagged sequences also may contain an 8His tag to produce a FLAG/8his tag attached to the fusion protein (DYKDDDDKHHHHHHHH, SEQ ID NO:12).

An example of a preferred fusion protein of the present invention is one in which NusA is fused to either a partial or a full-length mouse Notch-1 protein that comprises the 1731/1732 and 1743/1744 γ-secretase cleavage sites together with a short C-terminal FLAG/8His-tagged tail. The sequence of an exemplary fusion polypeptide is comprises amino acids 1703-1860 of mouse Notch (Le., depicted in SEQ ID NO:10) fused to a NusA protein. This exemplary fusion polypeptide has the sequence of SEQ ID NO:2. In order to monitor cleavage of this chimeric construct by γ-secretase, anti-Val 1744 antibodies against the Notch cleavage product containing Val-1744 residue are employed to determine the presence and concentration of the Val-1744 fragment generated as a result of Notch cleavage. Anti-Flag antibodies may be employed to detect the C-terminus of the chimeric construct. However, it should be noted that the chimeric construct may also employ a reporter protein such as alkaline phosphatase, for example, at the C-terminus instead of the flag tag. Such a reporter protein would be released as a result of the Notch cleavage and detected using assays well known to those of skill in the art.

In addition to providing fusion polypeptides as already described, the invention provides Notch fusion proteins that are further modified to incorporate, for example, a label or other detectable moiety.

For example, the Notch/NusA fusion protein substrates may comprise internally quenched labels that result in increased detectability after cleavage of the Notch substrate. The Notch/NusA fusion protein substrates may be modified to have attached a paired flurophore and quencher including but not limited to 7-amino, 4-methyl coumarin and 2,4-dinitrophenol, respectively, such that cleavage of the peptide by the γ-secretase results in increased fluorescence due to physical separation of the flurophore and quencher, which are attached on opposite sides of the scissile bond for the γ-secretase. Other paired flurophores and quenchers include bodipy-tetramethylrhodamine and QSY-5 (Molecular Probes, Inc.) In a variant of this assay, biotin or another suitable tag may be placed on one end of the peptide to anchor the peptide to a substrate assay plate and a flurophore may be placed at the other end of the fusion protein. Useful flurophores include those listed above as well as Europium labels such as W8044 (EG&g Wallac, Inc.) Another preferred label that may be used is Oregon green that may be attached to a Cys residue. Cleavage of the fusion protein by γ-secretase will release the flurophore or other tag from the plate, allowing compounds to be assayed for inhibition of proteolytic cleavage as shown by an increase in retained fluorescence. Preferred colorimetric assays of γ-secretase proteolytic activity utilize other Notch/NusA fusion proteins in which the amino acids comprising the γ-secretase recognition site for cleavage are linked to o-nitrophenol through an amide linkage, such that cleavage of the fusion protein by the γ-secretase results in an increase in optical density after altering the assay buffer to alkaline pH.

Further, the Notch/NusA fusion proteins may be labeled using labels well known to those of skill in the art, e.g., biotin labels are particularly contemplated. The use of such labels is well known to those of skill in the art and is described in, e.g., U.S. No. Pat. 3,817,837; U.S. Pat. No. 3,850,752; U.S. Pat. No. 3,996,345 and U.S. Pat. No. 4,277,437. Other labels that will be useful include but are not limited to radioactive labels, fluorescent labels and chemiluminescent labels. U.S. patents concerning use of such labels include, for example, U.S. Pat. No. 3,817,837; U.S. Pat. No. 3,850,752; U.S. Pat. No. 3,939,350 and U.S. Pat. No. 3,996,345. Any of Notch/NusA fusion protein compositions of the present invention may comprise one, two, or more of any of these labels.

### II. γ-SECRETASE COMPOSITIONS

In addition to novel Notch fusion proteins, the present invention is directed to methods of using such Notch fusion proteins in various γ-secretase assays. The present section provides a discussion of generating fractions containing proteins that have a γ-secretase activity.

While the exact identity of γ-secretase remains elusive there is strong evidence that γ-secretase may be presenilin 1. Regardless of the fact that the sequence of the γ-secretase protein has yet to be identified, those of skill in the art are aware of methods and compositions for isolating cellular fractions that comprises γ-secretase. For example, Li et al. (Proc. Natl. Acad. Sci. 97:6138-6143, 2000) described methods and compositions for producing a membrane preparation which contains a solubilized γ-secretase activity. Such a method is useful in the present invention for providing a purified fraction containing a γ-secretase. Once such a fraction is produced, it is contemplated that it may be used in the assays of the present invention. In addition, the novel fusion proteins of the present invention also may be used to further isolate and purify the γ-secretase from such a membrane fraction using, e.g., affinity chromatographic separation techniques.

The γ-secretase fraction generally is isolated from any cell that expresses a γ-secretase activity. For example, HeLa3 cells may be used. The cells are ruptured using e.g., a French press or other cell rupture technique, including but not limited to, freeze-thaw techniques (e.g., cycling cells between dry ice and 37° C. water bath); solid shear methods using a Hughes or French press; detergent lysis (e.g., on-ionic detergent solutions such as Tween, Triton, NP-40, etc.); hypotonic solution lysis (e.g., water, citric buffer); liquid shear methods (homogenizer; impinging-jet microfluidizer); sonication (ultrasound). After cell lysis, the cell debris and nuclei can be removed by sedimentation using centrifugation. The membrane fraction is precipitated at e.g., 100,000 g for 60 minutes and the membrane fraction may be further solubilized using detergent. For example, in the solubilization process taught by Li et al supra, the membrane fraction pellet from the 100,000 g centrifugation step is resuspended in buffer and treated with 1% CHAPSO for 60 minutes at 4° C. and centrifuged for 60 minutes. This detergent solubilization process is such that the supernatant of the 100,000 g fraction contains the solubilized γ-secretase.

The above solubilized fraction is used in the γ-secretase assays described herein throughout.

### III. PROTEIN OR PEPTIDE PRODUCTION AND PURIFICATION

The present invention provides soluble Notch protein substrates for use in the identification of modulators of γ-secretase that are specific for APP cleavage but do not inhibit the cleavage of Notch. Such substrates may be produced by conventional automated peptide synthesis methods or by recombinant expression.

A. Synthetic Peptide Production

The peptides or indeed even the full length fusion polypeptides of the invention can be synthesized in solution or on a solid support in accordance with conventional techniques. Various automatic synthesizers are commercially available and can be used in accordance with known protocols. See, for example, Stewart and Young, Solid Phase Peptide Synthesis, 2d. ed., Pierce Chemical Co., (1984); Tam et al., J. Am. Chem. Soc., 105:6442, 1983; Merrifield, Science, 232:341-347, 1986; and Barany and Merrifield, The Peptides, Gross and Meienhofer, eds, Academic Press, New York, 1-284, 1979, each incorporated herein by reference. The novel Notch fusion protein substrates of the invention comprise the γ-secretase cleavage sites 1731/1732 and 1743/1744 that are amenable to cleavage by γ-secretase can be readily synthesized and then screened in γ-secretase screening assays.

In particularly preferred methods, the fusion proteins of the present invention were synthesized by solid-phase technology employing a Model 433A from Applied Biosystems Inc. The purity of any given Notch fusion protein, generated through automated peptide synthesis or through recombinant methods may be determined using reverse phase HPLC analysis. Chemical authenticity of each peptide may be established by any method well known to those of skill in the art. In preferred embodiments, the authenticity is established by mass spectrometry.

Additionally, the fusion proteins may be quantitated using amino acid analysis in which microwave hydrolyses are conducted. Such analyses may use a microwave oven such as the CEM Corporation's MDS 2000 microwave oven. The peptide (approximately 2 mg protein) is contacted with 6 N HCl (Pierce Constant Boiling e.g., about 4 ml) with approximately 0.5% (volume to volume) phenol (Mallinckrodt). Prior to the hydrolysis, the samples are alternately evacuated and flushed with N2. The protein hydrolysis is conducted using a two-stage process. During the first stage, the fusion proteins are subjected to a reaction temperature of about 100° C. and held that temperature for 1 minute. Immediately after this step, the temperature is increased to 150° C. and held at that temperature for about 25 minutes. After cooling, the samples are dried and amino acid from the hydrolysed fusion proteins samples are derivatized using 6-aminoquinolyl-N-hydroxysuccinim idyl carbamate to yield stable ureas that fluoresce at 395 nm (Waters Acc·Tag Chemistry Package). The samples may be analyzed by reverse phase HPLC and quantification may be achieved using an enhanced integrator.

B. Recombinant Protein Production

As an alternative to automated peptide synthesis, recombinant DNA technology may be employed wherein a nucleotide sequence which encodes a peptide of the invention is inserted into an expression vector, transformed or transfected into an appropriate host cell and cultivated under conditions suitable for expression as described herein below. Recombinant methods are especially preferred for producing longer polypeptides that comprise peptide sequences of the invention.

From the disclosure of novel Notch fusion protein substrates of the present invention, it is possible to produce the fusion polypeptides by recombinant techniques. A variety of expression vector/host systems may be utilized to contain and express the peptide or fusion polypeptide coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the Notch fusion proteins in bacteria, yeast and other invertebrates are described herein below.

Expression vectors for use in prokaryotic hosts generally comprise one or more phenotypic selectable marker genes. Such genes generally encode, e.g., a protein that confers antibiotic resistance or that supplies an auxotrophic requirement. A wide variety of such vectors are readily available from commercial sources. Examples include pSPORT vectors, pGEM vectors (Promega), pPROEX vectors (LTI, Bethesda, Md.), Bluescript vectors (Stratagene), pET vectors (Novagen) and pQE vectors (Qiagen). The DNA sequence encoding the given Notch fusion protein is amplified by PCR and cloned into such a vector, for example, pGEX-3X (Pharmacia, Piscataway, N.J.) designed to produce a fusion protein comprising glutathione-S-transferase (GST), encoded by the vector, and a protein encoded by a DNA fragment inserted into the vector's cloning site. The primers for the PCR may be generated to include for example, an appropriate cleavage site. Treatment of the recombinant fusion protein with thrombin or factor Xa (Pharmacia, Piscataway, N.J.) is expected to cleave the fusion protein, releasing the substrate or substrate containing polypeptide from the GST portion. The pGEX-3X/fusion peptide construct is transformed into E. coli XL-1 Blue cells (Stratagene, La Jolla Calif.), and individual transformants were isolated and grown. Plasmid DNA from individual transformants is purified and partially sequenced using an automated sequencer to confirm the presence of the desired peptide or polypeptide encoding nucleic acid insert in the proper orientation.

The induction of the GST/substrate fusion protein is achieved by growing the transformed XL-1 Blue culture at 37° C. in LB medium (supplemented with carbenicillin) to an optical density at wavelength 600 nm of 0.4, followed by further incubation for 4 hours in the presence of 0.5 mM Isopropyl*-D-Thiogalactopyranoside (Sigma Chemical Co., St. Louis Mo.).

The GST fusion protein, expected to be produced as an insoluble inclusion body in the bacteria, may be purified as follows. Cells are harvested by centrifugation; washed in 0.15 M NaCl, 10 mM Tris, pH 8, 1 mM EDTA; and treated with 0.1 mg/ml lysozyme (Sigma Chemical Co.) for 15 minutes at room temperature. The lysate is cleared by sonication, and cell debris is pelleted by centrifugation for 10 minutes at 12,000×g. The fusion protein-containing pellet is resuspended in 50 mM Tris, pH 8, and 10 mM EDTA, layered over 50% glycerol, and centrifuged for 30 min. at 6000×g. The pellet is resuspended in standard phosphate buffered saline solution (PBS) free of Mg++ and Ca++. The fusion protein is further purified by fractionating the resuspended pellet in a denaturing SDS polyacrylamide gel (Sambrook et al. eds. Cold Spring Harbor Press, Cold Spring Harbor, N.Y. 1989). The gel is soaked in 0.4 M KCl to visualize the protein, which is excised and electroeluted in gel-running buffer lacking SDS. If the GST/Notch fusion protein is produced in bacteria as a soluble protein, it may be purified using the GST Purification Module (Pharmacia Biotech).

The fusion protein may be subjected to thrombin digestion to cleave the GST from the mature Notch fusion polypeptide. The digestion reaction (20-40 μg fusion protein, 20-30 units human thrombin (4000 U/mg (Sigma) in 0.5 ml PBS) is incubated 16-48 hrs. at room temperature and loaded on a denaturing SDS-PAGE gel to fractionate the reaction products. The gel is soaked in 0.4 M KCl to visualize the protein bands. The identity of the protein band corresponding to the expected molecular weight of the fusion polypeptide may be confirmed by partial amino acid sequence analysis using an automated sequencer (Applied Biosystems Model 473A, Foster City, Calif.).

Alternatively, the DNA sequence encoding the predicted substrate containing fusion polypeptide may be cloned into a plasmid containing a desired promoter and, optionally, a leader sequence (see, e.g., Better et al., Science, 240:1041-43, 1988). The sequence of this construct may be confirmed by automated sequencing. The plasmid is then transformed into E. coli using standard procedures employing CaCl₂ incubation and heat shock treatment of the bacteria (Sambrook et al., supra). The transformed bacteria are grown in LB medium supplemented with carbenicillin, and production of the expressed protein is induced by growth in a suitable medium. If present, the leader sequence will effect secretion of the mature Notch-based fusion protein and be cleaved during secretion.

The secreted recombinant protein is purified from the bacterial culture media by the method described herein throughout.

Similarly, yeast host cells from genera including Saccharomyces, Pichia, and Kluveromyces may be employed to generate the recombinant peptide. Preferred yeast hosts are S. cerevisiae and P. pastoris. Yeast vectors will often contain an origin of replication sequence from a 2T yeast plasmid, an autonomously replicating sequence (ARS), a promoter region, sequences for polyadenylation, sequences for transcription termination, and a selectable marker gene. Vectors replicable in both yeast and E. coli (termed shuttle vectors) may also be used. In addition to the above-mentioned features of yeast vectors, a shuttle vector will also include sequences for replication and selection in E. coli. Direct secretion of polypeptides expressed in yeast hosts may be accomplished by the inclusion of nucleotide sequence encoding the yeast I-factor leader sequence at the 5′ end of the substrate-encoding nucleotide sequence.

Generally, a given substrate may be recombinantly expressed in yeast using a commercially available expression system, e.g., the Pichia Expression System (Invitrogen, San Diego, Calif.), following the manufacturer's instructions. This system also relies on the pre-pro-alpha sequence to direct secretion, but transcription of the insert is driven by the alcohol oxidase (AOX1) promoter upon induction by methanol.

The secreted recombinant substrate is purified from the yeast growth medium by, e.g., the methods used to purify substrate from bacterial and mammalian cell supernatants.

Alternatively, a synthetic DNA encoding the novel substrate of the invention may be cloned into the baculovirus expression vector pVL1393 (PharMingen, San Diego, Calif.; Luckow and Summers, Bio/Technology 6:47 (1988)). This substrate-containing vector is then used according to the manufacturer's directions (PharMingen) to infect Spodoptera frugiperda cells in sF9 protein-free media and to produce recombinant protein. The protein or peptide is purified and concentrated from the media using a heparin-Sepharose column (Pharmacia, Piscataway, N.J.) and sequential molecular sizing columns (Amicon, Beverly, Mass.), and resuspended in PBS. SDS-PAGE analysis shows a single band and confirms the size of the protein, and Edman sequencing on a Porton 2090 Peptide Sequencer confirms its N-terminal sequence.

Alternatively, the Notch fusion protein substrate may be expressed in an insect system. Insect systems for protein expression are well known to those of skill in the art. In one such system, Autographa califomica nuclear polyhedrosis virus (AcNPV) is used as a vector to express foreign genes in Spodoptera frugiperda cells or in Trichoplusia larvae. The substrate coding sequence is cloned into a nonessential region of the virus, such as the polyhedrin gene, and placed under control of the polyhedrin promoter. Successful insertion of substrate will render the polyhedrin gene inactive and produce recombinant virus lacking coat protein coat. The recombinant viruses are then used to infect S. frugiperda cells or Trichoplusia larvae in which the substrate is expressed (Smith et al., J Virol 46:584, 1983; Engelhard E K et al., Proc. Nat. Acad. Sci. 91:3224-7, 1994).

Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

It is preferable that the transformed cells are used for long-term, high-yield protein production and as such stable expression is desirable. Once such cells are transformed with vectors that contain selectable markers along with the desired expression cassette, the cells may be allowed to grow for 1-2 days in an enriched media before they are switched to selective media. The selectable marker is designed to confer resistance to selection and its presence allows growth and recovery of cells which successfully express the introduced sequences. Resistant clumps of stably transformed cells can be proliferated using tissue culture techniques appropriate to the cell.

A number of selection systems may be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk-, hgprt- or aprt- cells, respectively. Also, anti-metabolite resistance can be used as the basis of selection for dhfr, which confers resistance to methotrexate; gpt that confers resistance to mycophenolic acid; neo that confers resistance to the aminoglycoside G418; ALS which confers resistance to chlorsulfuron; and hygro that confers resistance to hygromycin. Additional selectable genes that may be useful include trpB, which allows cells to utilize indole in place of tryptophan, or hisD, which allows cells to utilize histinol in place of histidine. Markers that give a visual indication for identification of transformants include anthocyanins, b-glucuronidase and its substrate, GUS, and luciferase and its substrate, luciferin.

C. Site-Specific Mutagenesis

Site-specific mutagenesis is another technique useful in the preparation of individual γ-secretase substrate peptide and more particularly fusion polypeptides that comprise as a component one of the γ-secretase substrate fusion proteins of the present invention. This technique employs specific mutagenesis of the underlying DNA (that encodes the amino acid sequence that is targeted for modification). The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids also are routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization (annealing) conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

Of course, the above described approach for site-directed mutagenesis is not the only method of generating potentially useful mutant peptide species and as such is not meant to be limiting. The present invention also contemplates other methods of achieving mutagenesis such as for example, treating the recombinant vectors carrying the gene of interest mutagenic agents, such as hydroxylamine, to obtain sequence variants.

D. Protein Purification

It will be desirable to purify the fusion proteins of the present invention. Protein purification techniques are well known to those of skill in the art. These techniques involve, at one level, the crude fractionation of the cellular milieu to polypeptide and non-polypeptide fractions. Having separated the peptide or polypeptides of the invention from other proteins, the fusion polypeptides or peptides of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying fusion proteins is fast protein liquid chromatography (FPLC) or even high performance liquid chromatography (HPLC). In particularly preferred embodiments, the NusA-Notch fusion was isolated using immobilized metal affinity chromatography (IMAC).

IMAC is used primarily in the purification of polyhistidine tagged recombinant proteins. In the present invention the C-terminus of the fusion protein comprises a polyhistidine tag, thereby allowing purification through this powerful technique. This purification relies on the natural tendency of histidine to chelate divalent metals. Placing the metal ion on a chromatographic support allows purification of the histidine tagged proteins. This is a highly efficient method that has been employed by those of skill in the art for a variety of protein purification methods. Exemplary conditions of a preferred embodiment of isolating the protein of the present invention are described in Example 1. However, it should be understood that those of skill in the art could vary the conditions and media and still achieve purification in accordance with the present invention. To this end, those of skill in the art are referred to U.S. Pat. No. 4,431,546 which describes in detail methods of metal affinity chromatographic separation of biological or related substances from a mixture. The chromatographic media described in the aforementioned patent comprise binding materials which have a ligand containing at least one of the groups anthraquinone, phthalocyanine or aromatic azo, in the presence of at least one metal ion selected from the group Ca²⁺, Sr²⁺, Ba²⁺, Al³⁺, Co²⁺, Ni²⁺, Cu²⁺ or Zn²⁺. Additonal media and conditions are described at e.g., http://www.affiland.com/imac/nta.htm and http://www.affiland.com/imac/pdc.htm.

Certain aspects of the present invention concern the purification, and in particular embodiments, the substantial purification, of an encoded polypeptide, protein or peptide. The term “purified polypeptide, protein or peptide” as used herein, is intended to refer to a composition, isolated from other components, wherein the polypeptide, protein or peptide is purified to any degree relative to the cellular or synthesis components used to generate the protein. A purified polypeptide, protein or peptide therefore also refers to a polypeptide, protein or peptide, free from the environment in which it may naturally occur.

Generally, “purified” will refer to a polypeptide, protein or peptide composition that has been subjected to fractionation to remove various other components, and which composition substantially retains its expressed biological activity. Where the term “substantially purified” is used, this designation will refer to a composition in which the polypeptide, protein or peptide forms the major component of the composition, such as constituting about 50%, about 60%, about 70%, about 80%, about 90%, about 95% or more of the proteins in the composition.

Various methods for quantifying the degree of purification of the polypeptide, protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed polypeptide, protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified polypeptide, protein or peptide.

There is no general requirement that the polypeptide, protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., Biochem. Biophys. Res. Comm., 76:425, 1977). It will therefore be appreciated that under differing electrophoresis conditions, the apparent molecular weights of purified or partially purified expression products may vary.

### IV. EXPRESSION CONSTRUCTS FOR USE IN THE PRODUCTION OF SUBSTRATES OF THE INVENTION

In the present invention, it may be necessary to express the Notch fusion proteins of the present invention. To achieve such expression, the present invention will employ vectors comprising polynucleotide molecules for encoding the Notch fusion proteins of the present invention, as well as host cell transformed with such vectors. Such polynucleotide molecules may be joined to a vector, which generally includes a selectable marker and an origin of replication, for propagation in a host. These elements of the expression constructs used in the present invention are described in further detail herein below.

The expression vectors include DNA encoding any of the given peptide or fusion polypeptide γ-secretase substrates described above or below, operably linked to suitable transcriptional or translational regulatory sequences, such as those derived from a mammalian, microbial, viral, or insect gene. Examples of regulatory sequences include transcriptional promoters, operators, or enhancers, mRNA ribosomal binding sites, and appropriate sequences which control transcription and translation.

The terms “expression vector,” “expression construct ” or “expression cassette ” are used interchangeably throughout this specification and are meant to include any type of genetic construct containing a nucleic acid coding for a gene product in which part or all of the nucleic acid encoding sequence is capable of being transcribed.

The choice of a suitable expression vector for expression of the fusion polypeptides of the invention will of course depend upon the specific host cell to be used, and is within the skill of the ordinary artisan. Examples of suitable expression vectors include pcDNA3 (Invitrogen) and pSVL (Pharmacia Biotech). A preferred vector for expression in the present invention is pcDNA3.1-Hygro (Invitrogen). Expression vectors for use in mammalian host cells may include transcriptional and translational control sequences derived from viral genomes. Commonly used promoter sequences and enhancer sequences which may be used in the present invention include, but are not limited to, those derived from human cytomegalovirus (CMV), Adenovirus 2, Polyoma virus, and Simian virus 40 (SV40). Methods for the construction of mammalian expression vectors are disclosed, for example, in Okayama and Berg (Mol. Cell. Biol. 3:280, 1983); Cosman et al. (Mol. Immunol. 23:935, 1986); Cosman et al. (Nature 312:768, 1984); EP-A-0367566; and WO 91/18982.

The expression construct will comprise a nucleic acid region that encodes the particular Notch fusion proteins of the present invention. Coding regions for use in constructing such expression vectors should encode at least the γ-secretase cleavage of the fusion proteins described herein although it is contemplated that larger polypeptides may be encoded as long as one of the peptide generated comprises γ-secretase cleavage sites 1731/1732 and 1743/1744 that are amenable to cleavage by γ-secretase.

In certain aspects of the present invention, the expression construct may further comprise a selectable marker that allows for the detection of the expression of the peptide or polypeptide. Usually the inclusion of a drug selection marker aids in cloning and in the selection of transformants, for example, neomycin, puromycin, hygromycin, DHFR, zeocin and histidinol. Alternatively, enzymes such as herpes simplex virus thymidine kinase (tk) (eukaryotic), b-galactosidase, luciferase, or chloramphenicol acetyltransferase (CAT) (prokaryotic) may be employed. Immunologic markers also can be employed. For example, epitope tags such as the FLAG system (IBI, New Haven, Conn.), HA and the 6xHis system (Qiagen, Chatsworth, Calif.) may be employed. Additionally, glutathione S-transferase (GST) system (Pharmacia, Piscataway, N.J.), or the maltose binding protein system (NEB, Beverley, Mass.) also may be used. The selectable marker employed is not believed to be important, so long as it is capable of being expressed simultaneously with the nucleic acid encoding a gene product. Further examples of selectable markers are well known to one of skill in the art. Particularly preferred selectable markers that may be employed in the present invention are neomycin resistance or a GFP marker.

Expression requires that appropriate signals be provided in the vectors. The present section includes a discussion of various regulatory elements, such as enhancers/promoters from both viral and mammalian sources that may be used to drive expression of the nucleic acids of interest in host cells. Elements designed to optimize messenger RNA stability and translatability in host cells also are defined. The conditions for the use of a number of dominant drug selection markers for establishing permanent, stable cell clones expressing the products also are provided, as is an element that links expression of the drug selection markers to expression of the mutant phenotype.

In preferred embodiments, the nucleic acid encoding the given peptide or the nucleic acid encoding a selectable marker is under transcriptional control of a promoter. A “promoter” refers to a DNA sequence recognized by the synthetic machinery of the cell, or introduced synthetic machinery, required to initiate the specific transcription of a gene.

Nucleotide sequences are operably linked when the regulatory sequence functionally relates to the DNA encoding the Notch fusion protein. Thus, a promoter nucleotide sequence is operably linked to a given DNA sequence if the promoter nucleotide sequence directs the transcription of the sequence. Similarly, the phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 by of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 by upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 by apart before activity begins to decline. Depending on the promoter, it appears that individual elements can function either co-operatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter, the phosphoglycerol kinase promoter and glyceraldehyde-3-phosphate dehydrogenase promoter, all of which are promoters well known and readily available to those of skill in the art, can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well known properties, the level and pattern of expression of the protein of interest following transfection or transformation can be optimized.

Selection of a promoter that is regulated in response to specific physiologic or synthetic signals can permit inducible expression of the gene product. Several inducible promoter systems are available for production of viral vectors. One such system is the ecdysone system (Invitrogen, Carlsbad, Calif.), which is designed to allow regulated expression of a gene of interest in mammalian cells. It consists of a tightly regulated expression mechanism that allows virtually no basal level expression of the transgene, but over 200-fold inducibility.

Another useful inducible system is the Tet-Off™ or Tet-On™ system (Clontech, Palo Alto, Calif.) originally developed by Gossen and Bujard (Gossen and Bujard, Proc. Natl. Acad. Sci. U.S.A. 15;89(12):5547-51, 1992; Gossen et al., Science, 268(5218):1766-9, 1995).

In mammalian cells, the CMV immediate early promoter if often used to provide strong transcriptional activation. Modified versions of the CMV promoter that are less potent have also been used when reduced levels of expression of the transgene are desired. Retroviral promoters such as the LTRs from MLV or MMTV are contemplated to be useful in the present invention. Other viral promoters that may be used include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

In some embodiments, regulatable promoters may prove useful. Such promoters include for example, those that are hormone or cytokine regulatable. Hormone regulatable promoters include MMTV, MT-1, ecdysone and RuBisco as well as other hormone regulated promoters such as those responsive to thyroid, pituitary and adrenal hormones.

Another regulatory element contemplated for use in the present invention is an enhancer. These are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization. Enhancers useful in the present invention are well known to those of skill in the art and will depend on the particular expression system being employed (Scharf D et al., (1994) Results Probl Cell Differ 20: 125-62; Bittner et al., (1987) Methods in Enzymol. 153: 516-544).

Where an expression construct employs a cDNA insert, one will typically desire to include a polyadenylation signal sequence to effect proper polyadenylation of the gene transcript. Any polyadenylation signal sequence recognized by cells of the selected transgenic animal species is suitable for the practice of the invention, such as human or bovine growth hormone and SV40 polyadenylation signals.

Also contemplated as an element of the expression cassette is a terminator. These elements can serve to enhance message levels and to minimize read through from the cassette into other sequences. The termination region which is employed primarily will be one selected for convenience, since termination regions for the applications such as those contemplated by the present invention appear to be relatively interchangeable. The termination region may be native with the transcriptional initiation, may be native to the DNA sequence of interest, or may be derived for another source.

In certain embodiments of the invention, the use of internal ribosome entry site (IRES) elements is contemplated to create multigene, or polycistronic, messages. IRES elements are able to bypass the ribosome scanning model of 5′ methylated Cap dependent translation and begin translation at internal sites (Pelletier and Sonenberg, Nature, 334:320-325, 1988). IRES elements from two members of the picornavirus family (poliovirus and encephalomyocarditis) have been described (Pelletier and Sonenberg, 1988 supra), as well an IRES from a mammalian message (Macejak and Sarnow, Nature, 353:90-94, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

### V. USE OF THE SUBSTRATES IN γ-SECRETASE ASSAYS

In specific embodiments, the present invention involves assays to monitor the activity and/or function of γ-secretase and more specifically, the γ-secretase activity and/or function of γ-secretase. These assays will involve incubating in solution a γ-secretase complex (or purified polypeptide) with a suitable substrate of the present invention, using cleavage of the Notch fusion protein as a measure of γ-secretase proteolytic activity.

A. Assay Formats

In specific embodiments, the invention relates to a method for the identification of agents that modulate the activity of human γ-secretase. An aspect of these assays is to monitor the Notch-cleaving activity of the γ-secretase at the ε cleavage site 1743/1744 in the presence and absence of the putative modulator compound or agent. For example, such a method for determining Notch cleavage would generally comprise the steps of:

(a) contacting any of the Notch/NusA fusion protein of the present invention in vitro with a composition comprising a γ-secretase activity;

(b) determining the cleavage of the Notch/NusA fusion protein at the γ-secretase cleavage site of Notch by said γ-secretase.

The composition comprising γ-secretase activity would generally be any isolated composition that comprises a γ-secretase activity. As such, the composition may be a membrane fraction isolated from a cell (a natural cell that has γ-secretase activity, or a recombinant host cell that has been engineered to express such an activity). Alternatively, composition may comprise an isolated and purified γ-secretase protein, substantially free of other proteins.

In order to identify modulators of the Notch cleavage activity of γ-secretase, the above steps (a) and (b) are carried out in the presence and absence of the candidate modulator, and the modulating activity of the modulator is assessed by comparing the Notch cleavage activity of the γ-secretase in the presence of the test agent to the activity in the absence of the test agent to identify an agent that modulates such cleavage by the γ-secretase. Any alteration in the amount or degree of Notch cleavage in the presence of the candidate modulator is an indication of an alteration of the γ-secretase activity in the presence of the test agent identifies an agent that is a modulator of the γ-secretase activity.

Agents that cause increased cleavage relative to the control (no test agent) are scored as agonists or stimulators of γ-secretase proteolytic activity, whereas agents to cause decreased cleavage at either Aβ 1-40 and/or Aβ 1-42 are scored as inhibitors. The inhibitors of γ-secretase are of special interest because inhibitors of γ-secretase activity have therapeutic and prophylactic indications for the treatment and prevention of AD or its symptoms or progression.

Because it is desirable to find test compounds that preferentially inhibits gamma secretase mediated cleavage of a APP or CT-100 compared to cleavage of a fusion protein of the invention, test compounds may be assessed to determine their ability to modulate APP cleavage in parallel or prior to utilizing the assays of the invention. Methods of measuring gamma secretase mediated APP or CT-100 cleavage are well known in the art. As an example, WO/01/83811 teaches substrates and in-vitro assays useful in measuring APP or CT-100 cleavage. WO/01/83811 teaches a gamma-secretase substrate comprising an M terminal Met (M), APP597-695 and a Flag tag and methods and conditions for its cleavage and detection of the cleavage products (M-Aβ40 and M-Aβ40).

Inhibitors that inhibit the APP/CT-100 cleavage activity at either Aβ 1-40 and/or Aβ 1-42 but do not inhibit the measured Notch cleavage activity of the γ-secretase are most preferred. The γ-secretase may be a purified γ-secretase polypeptide or complex or biologically active fragments, analogs, or variants, thereof. In preferred embodiments, the γ-secretase is derived from a membrane fraction from a cell that exhibits γ-secretase activity. Preferably, such a membrane fraction contains all the components needed for the γ-secretase complex. Non-human orthologs of human γ-secretase also may be used in assays.

The assays of the present invention are designed to be performed with γ-secretase polypeptide in a cell free system. For example, in a cell-free system, the contacting step may be performed by mixing the γ-secretase enzyme or a membrane fraction containing that enzyme with the peptide or protein substrate of the invention, in the presence or absence of the test agent. For optimal results, the enzyme and the γ-secretase substrate preferably are substantially purified, mixed in defined and controlled quantities, and mixed in appropriate buffers that optimize enzymatic activity and/or mimic physiological conditions.

The determining step may involve a measurement of an N-terminal fragment, a C-terminal fragment, or both, or may involve measurement of another parameter indicative of cleavage. For example, the Notch-based or other γ-secretase substrate may contain a quenched label that becomes more detectable only upon cleavage to separate the label from the quenching moiety. Alternatively, the Notch-based or other γ-secretase substrate may be fixed at the N-terminal or C-terminal end to a solid support. In this arrangement, cleavage may be measured by release from the solid support of a cleavage fragment. The release may be measured by increased label in the media, or decreased label attached to the solid support. Alternatively, the release may be measured by quantitative capture of the released peptide (e.g., with an antibody).

In a preferred embodiment of the present invention, the cleaved Notch protein is detected using an antibody specific for Val-1744. In even more preferred embodiments, the cleaved Notch protein was detected using an ELISA developed based on anti-Val 1744 antibody and anti-Flag antibodies. The anti-Val 1744 antibodies are used to detect one fragment of the cleavage, whereas the anti-Flag antibodies detect the fragment (C-terminal) of the Notch protein which produced by the action of the γ-secretase enzyme. This assay is described in further detail in the Examples.

Of course, the above assay is only exemplary and other assays also may be used. For example, the above assay may be set up in the following manner. 384-well micro-titer plates are blocked with BSA, γ-secretase enzyme and 50 μM of the γ-secretase inhibitor compound to be tested are incubated for 1 hour and the reaction is initiated by the addition of Notch/NusA fusion protein substrate. In the final assay conditions, the volume is 30 μl/well; 50 μM compound; 15 ng enzyme/well; 250 nM substrate; 5% DMSO and 0.001% TWEEN-20. The assay is incubated overnight at room temperature and the reaction is terminated by the addition of Tris-HCl, pH 8.3. An aliquot containing 6.25 pmoles of substrate is removed and the cleaved and/or uncleaved substrate is captured in a streptavidin coated plate. The plate is washed 3 time and buffer is added. The capture assay is monitored by reading the fluorescence emission of the oregon green on an LJL Analyst (Ex 485/Em 530).

Another assay that may be used herein is a fluorescent polarization assay. Fluorescence polarization is a sensitive, facile and non-destructive assay that can be exploited to monitor the effects of the candidate agents on the γ-secretase-complex of the present invention. It can be used to monitor the interaction of these substrates with the γ-secretase enzyme. Under controlled conditions, fluorescence polarization measurements can reveal the extent of “molecular tumbling” of a fluorescent molecule in solution. For example, a small molecule with a compact molecular volume would be expected to tumble rapidly. If irradiated with polarized light the rapid movement of the molecule in solution would result in extensive depolarization of the light, and would yield a readout of “low” polarization value. Under the same conditions, the increased molecular volume of a large molecule or a large complex would slow the molecular rotation (tumbling) process. As a result, less polarization of the incident plane polarized light would result and a higher polarization value would be measured.

By labeling a small ligand with a fluorescent probes, changes in the fluorescence polarization resulting from the interaction of the ligand with another system component can be measured. Such a method may be applied to measure the strength of interaction between an enzyme (γ-secretase) and a fluorescent enzyme substrate.

In an exemplary fluorescence polarization assay, in pre-blocked low affinity, black plates enzyme and inhibitory/modulatory compound are incubated for 30 minutes and the reaction initiated by the addition of 150nM substrate (e.g., a fluorescently labeled Notch/NusA fusion protein of the present invention) to a final volume of 30 μl/well. The plate is then incubated at room temperature for 15 minutes and the fluorescent polarization measured on an LJL Acquest (Ex 485/Em 530).

An aspect of the present invention that would be useful in isolating and characterizing the γ-secretase is contemplated by the present invention. This aspect contemplates a binding assay for detecting compounds that bind to the active site or at an allosteric site of the enzyme. For such determinations, the use of non-hydrolyzable derivatives of the Notch substrates of the present invention may be used. For example, the presence of a statine derivative at the junction of the bond to be cleaved renders the Notch of the present invention non-hydrolyzable at the cleavage site. The substrates further may be modified with the addition of an appropriate fluorescent tag e.g., BODIPY FL to facilitate detection.

A substrate of the present invention may be labeled with a fluorescent label and used to develop a fluorescence polarization binding assay for the γ-secretase. The equilibrium dissociation constant (KD) for the interaction between the enzyme and the substrate is determined by measuring fluorescence polarization changes which result from titrating the substrate with the enzyme.

To determine the KD for the interaction of a substrate of the present invention with γ-secretase, various quantities of γ-secretase may be combined with 3.1 nM fluorescent substrate and incubated at room temperature for 3 hours. Following the incubation, fluorescence polarization is determined using an LJL Analyst (96 well format) or a PanVera Beacon (single cuvette format). An exemplary assay is performed in 25 mM sodium acetate, 20% glycerol, pH 4.75. A graphic plot of the data obtained providing the polarization values on the vertical axis and the concentration of enzyme on the horizontal axis provides the binding isotherm for the determination of the KD for the interaction of the enzyme with the substrate. The data may then be analyzed using the relation Px=PF+(PB-PF)*[E]/(KD+[E]), where P=polarization value, x=sample, F=free inhibitor, B=bound inhibitor, E=γ-secretase (Fluorescence Polarization Applications Guide, 1998; PanVera, Madison, Wis.) to obtain the KD. This assay can be used to screen for compounds that bind to the active site of the enzyme or allosterically.

It will be appreciated that the activity measurements in the presence and absence of a test agent can be performed in parallel, or sequentially, in either order. Moreover, it may not be necessary to repeat the control measurements (i.e., the measurements of cleavage in the absence of a test agent) in parallel with respect to every test agent, once a reliable baseline of enzymatic activity for particular reaction conditions has been obtained. Gained knowledge of the enzymatic activity of γ-secretase towards a particular substrate (e.g., the Notch compositions of the present invention or a composition derived from APP) in the absence of inhibitors can be used as the basis for performing the comparison step.

B. Candidate Substances

As used herein the term “candidate substance” or “test substance” refers to any molecule that is capable of modulating γ-secretase activity, and preferably human γ-secretase activity. The candidate substance may be a protein or fragment thereof, a small molecule inhibitor, or even a nucleic acid molecule. It may prove to be the case that the most useful pharmacological compounds for identification through application of the screening assay will be compounds that are identified through screening large compound libraries or that are structurally related to other known modulators of APP processing, Notch processing or both. For example, U.S. Pat. No. 6,448,229, incorporated herein by reference, describes a specific class of compounds that inhibit γ-secretase without affecting Notch signalling, and hence find use in the treatment or prevention of AD. Compounds such as those described in U.S. Pat. No. 6,448,229 may be verified using the assays of the present invention. In addition, such compounds may serve as starting materials in rational drug design to identify additional candidate modulators for use in the present invention. Other inhibitory agents that could be used for such rational drug design include but are not limited to DAPT (PHA-568638) and fenchylamine (PHA-512088).

The candidate substances may include fragments or parts of naturally-occurring compounds or may be only found as active combinations of known compounds which are otherwise inactive. However, prior to testing of such compounds in humans or animal models, it will be necessary to test a variety of candidates to determine which have potential.

Accordingly, the candidate substance may include fragments or parts of naturally-occurring compounds or may be found as active combinations of known compounds which are otherwise inactive. Accordingly, the present invention provides screening assays to identify agents which stimulate or inhibit γ-secretase-mediated cellular APP processing preferentially over the stimulation or inhibition of Notch by that enzyme. It is proposed that compounds isolated from natural sources, such as animals, bacteria, fungi, plant sources, including leaves and bark, and marine samples may be assayed as candidates for the presence of potentially useful pharmaceutical agents.

It will be understood that the pharmaceutical agents to be screened could also be derived or synthesized from chemical compositions or man-made compounds. Thus, it is understood that the modulator identified by the present invention may be polypeptide, polynucleotide, small molecule inhibitors or any other inorganic or organic chemical compounds that may be designed through rational drug design starting from known stimulators or inhibitors of γ-secretase activity and/or APP processing.

The candidate screening assays are simple to set up and perform. Thus, in assaying for a modulator, after obtaining a cell membrane fraction comprising a functional γ-secretase (e.g., a cell membrane fraction containing a solubilized γ-secretase complex), one will admix a candidate substance with such a γ-secretase composition in the presence of the novel substrates of the present invention, under conditions which would allow measurable γ-secretase activity, through cleavage of the substrate, to occur. In this fashion, one can measure the ability of the candidate substance to stimulate the activity of the γ-secretase in the absence of the candidate substance. Likewise, in assays for inhibitors after obtaining a cell membrane fraction expressing functional γ-secretase, the candidate substance is admixed with that fraction. In this fashion the ability of the candidate inhibitory substance to reduce, abolish, or otherwise diminish a biological effect mediated by γ-secretase may be detected.

“Effective amounts” of the substance in certain circumstances are those amounts effective to reproducibly alter a given CT-100 cleaving γ-secretase activity or APP processing. These effective amounts are those which alter the degree or amount of cleavage of the APP CT-100 but do not alter the cleavage of the Notch fusion proteins of the present invention at γ-secretase cleavage site in comparison to the cleavage seen in the absence of the candidate substance. Compounds that will be particularly useful as therapeutic agents and/or for further characterization, are those compounds that preferentially inhibit APP cleavage by the γ-secretase as compared to Notch cleavage. By “preferentially inhibit” it is meant that the agent has more of an inhibitory effect on γ-secretase mediated cleavage of APP than on Notch. While it would be preferable that the agent is one which has no inhibitory effect on the Notch cleavage, some inhibition of the Notch cleavage may be acceptable so long as it is less than the APP cleavage seen by the wild-type γ-secretase enzyme.

The assays described above employing the novel γ-secretase substrates of the invention are amenable to numerous high throughput screening (HTS) methods (For a review see Jayawickreme and Kost, Curr. Opin. Biotechnol. 8:629-634, 1997). Automated and miniaturized HTS assays are also contemplated as described for example in Houston and Banks Curr. Opin. Biotechnol. 8:734-740, 1997.

There are a number of different libraries used for the identification of small molecule modulators including chemical libraries, natural product libraries and combinatorial libraries comprised or random or designed peptides, oligonucleotides or organic molecules. Chemical libraries consist of structural analogs of known compounds or compounds that are identified as hits or leads via natural product screening or from screening against a potential therapeutic target. Natural product libraries are collections of products from microorganisms, animals, plants, insects or marine organisms which are used to create mixtures of screening by, e.g., fermentation and extractions of broths from soil, plant or marine organisms. Natural product libraries include polypeptides, non-ribosomal peptides and non-naturally occurring variants thereof. For a review see Science 282:63-68, 1998.

Combinatorial libraries are composed of large numbers of peptides oligonucleotides or organic compounds as a mixture. They are relatively simple to prepare by traditional automated synthesis methods, PCR cloning or other synthetic methods. Of particular interest will be libraries that include peptide, protein, peptidomimetic, multiparallel synthetic collection, recombinatorial and polypeptide libraries. A review of combinatorial libraries and libraries created therefrom, see Myers Curr. Opin. Biotechnol. 8:701-707, 1997. A modulator identified by the use of various libraries described may then be optimized to modulate activity of γ-secretase through, for example, rational drug design.

It will, of course, be understood that all the screening methods of the present invention are useful in themselves notwithstanding the fact that effective candidates may not be found. The invention provides methods for screening for such candidates, not solely methods of finding them.

C. In Vivo Assays

The present invention also encompasses the use of various animal models. Once the modulators have been screened in an in vitro environment as discussed above, any non-human models of APP processing and/or AD may be used to determine the in vivo effects of the modulators. This will afford an excellent opportunity to examine the function of γ-secretase in a whole animal system where it is normally expressed.

Treatment of animals with test compounds that have been identified as modulators of γ-secretase activity will involve the administration of the compound, in an appropriate form, to the animal. Administration will be by any route that can be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood, cerebrospinal fluid (CSF) or lymph supply and intratumoral injection.

Determining the effectiveness of a compound in vivo may involve a variety of different criteria. Such criteria include, but are not limited to, survival, increased activity level, and improved food intake. Other methods of evaluation include pathological examination, especially of brain tissue, to look for indicia of altered γ-secretase activity, such as reduced production of amyloid beta or amyloid beta plaques and reduced atrophy of the brain.

D. Manufacture of Medicaments

The assays of the invention will identify γ-secretase modulators that represent candidate therapeutics for treatment of diseases characterized by aberrant levels of γ-secretase activity, including AD. Thus, after identifying modulator agents, the methods of the invention optionally include the additional step or steps of manufacturing/synthesizing the agents, and of formulating the agent into a composition using pharmaceutically acceptable diluents, adjuvants, or carriers. Pharmaceutical compositions are described in greater detail below.

### VI. PHARMACEUTICAL COMPOSITIONS

The modulators of Notch processing APP processing, and/or γ-secretase cleavage identified by the present invention may ultimately be formulated into pharmaceutical compositions i.e., in a form appropriate for in vivo applications. Generally, this will entail preparing compositions that are essentially free of pyrogens, as well as other impurities that could be harmful to humans or animals.

One will generally desire to employ appropriate salts and buffers to render the identified modulator compositions stable and allow for their uptake by target cells. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the modulators identified by the present invention, its use in therapeutic compositions is contemplated. Supplementary active ingredients also can be incorporated into the compositions.

The modulator compositions of the present invention include classic pharmaceutical preparations. Administration of these compositions according to the present invention will be via any common route so long as the target tissue is available via that route. The pharmaceutical compositions may be introduced into the subject by any conventional method, e.g., by intravenous, intradermal, intramusclar, intramammary, intraperitoneal, intrathecal, intraocular, retrobulbar, intrapulmonary (e.g., term release); by oral, sublingual, nasal, anal, vaginal, or transdermal delivery, or by surgical implantation at a particular site, e.g., embedded under the splenic capsule, brain, or in the cornea. The treatment may consist of a single dose or a plurality of doses over a period of time.

The modulator compounds identified using the present invention may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

For oral administration the modulators identified by the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

“Unit dose” is defined as a discrete amount of a therapeutic composition dispersed in a suitable carrier. For example, parenteral administration may be carried out with an initial bolus followed by continuous infusion to maintain therapeutic circulating levels of drug product. Those of ordinary skill in the art will readily optimize effective dosages and administration regimens as determined by good medical practice and the clinical condition of the individual patient. More particularly, the dose should be selected to reduce, inhibit, decrease or otherwise abrogate the formation of Aβ-peptide and more particularly, plaque formation in the brain of a subject exhibiting AD. To this effect, those of skill in the art will be able to employ animal models of AD (e.g., as disclosed in U.S. Pat. No. 5,877,399; U.S. Pat. No. 5,387,742; U.S. Pat. No 5,811,633) in order to optimize dose administration protocols and predict the relevant amounts of pharmaceutical agents required for intervention of AD in a human subject.

The frequency of dosing will depend on the pharmacokinetic parameters of the agents and the routes of administration. The optimal pharmaceutical formulation will be determined by one of skill in the art depending on the route of administration and the desired dosage: See for example Remington's Pharmaceutical Sciences, 18th Ed. (1990, Mack Publ. Co, Easton Pa. 18042) pp 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Appropriate dosages may be ascertained through the use of established assays for determining blood levels in conjunction with relevant dose-response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey, ducks and geese.

### VI. EXAMPLES

The following examples are included to demonstrate preferred embodiments of the invention. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

The present Example provides a teaching of the general techniques, reagents and assays employed to obtain the results discussed herein. General laboratory chemicals were purchased from Sigma Chemical Co (St. Louis, Mo.). The pET 43.1a vector was from Novagen (Madison, Wis.) and restriction enzymes were from Invitrogen (Carlsbad, Calif.). Oligonucleotides were from Sigma Genosys (The Woodlands, Tex.). The Val-1744 antibody was from Cell Signaling Technology (Beverly, Mass.). PHA/PNU compounds were obtained from the Pharmacia compound collection (New Jersey, USA).

Cloning Notch Substrate: Notch substrate containing amino acids N1703-D1860 of mouse notch-1 protein (DNA Sequence accession number Z11886) was cloned into pET 43.1a vector as an EcoRI/HindIII insert in frame with a nucleic acid that encoded the NusA. The expression construct for encoding the NusA/Notch fusion protein had a sequence of SEQ ID NO:SEQ ID NO:1. This construct contained C-terminal Flag and 8His tags generated through PCR to give the extension (DYKDDDDKHHHHHHHH, SEQ ID NO:12) following amino acid 1860 of Notch. The DNA was transformed into BL21 (DE3) competent E. coli (Stratagene) and clones were screened for the presence of the correct insert using DNA Concert miniprep kit (Gibco/BRL). Clone Notch-F6 was found to contain the correct DNA sequence of SEQ ID NO:1.

Expression and Purification of Notch/NusA fusion protein. E. coli was transformed with clone Notch-F6 was inoculated into LB/Amp and grown overnight at 37° C. in a shaking incubator. The next day, 35 ml of the overnight culture was used to inoculate 2 liters of LB/Amp. The E. coli were grown at 37° C. in a shaking incubator until the A₆₀₀ reached 0.4 and then were induced with 1 mM IPTG for 3 hours and centrifuged. Pellets from two liters of culture were resuspended in 5 ml/g pellet of 50 mM Tris, pH 8.0, 100 mM NaCl including protease inhibitors and were processed three times with a French Press to yield a crude extract. The pH of the extract was adjusted to 8.0 using 2M Tris and was centrifuged at 11,000 g for 45 min. The supernatant was loaded onto a 4 ml nickel IMAC chromatography column equilibrated in 50 mM Tris, pH 8.0, 100 mM NaCl, and with protease inhibitors. Column was washed with the same buffer followed by buffer containing 50 mM imidazole. NusA-Notch fusion protein was eluted with buffer containing 300 mM imidazole and 0.8 ml fractions were collected and analyzed by A₂₈₀ and SDS-PAGE. Fractions containing NusA-Notch were pooled and dialyzed into 50 mM Pipes, pH 7.0, 100 mM NaCl.

Cleavage of Notch as Determined by Western Blot Analysis. NusA-Notch fusion (1.7 μM) was incubated with 70 mg/ml solubilized γ-secretase in 50 mM Pipes, pH 7.0, 0.25% CHAPSO in a total volume of 50 μl overnight at 37° C. DAPT (PHA-568638) was added at varying concentrations. The reactions were stopped with the addition of 12.5 μl of 5× Laemmli buffer (Laemmli 1970) and 30 μl of the mixture was electrophoresed on a 15% SDS-PAGE. Proteins were transferred to nitrocellulose using a semi-dry blot apparatus (Millipore) and blocked for 2 hours using 4% BSA in PBS/0.5% Tween-20. Val-1744 antibody was added to the blocking solution at a 1:1000 dilution and incubated for 1 hour. The membrane was washed three times with PBS/0.5% Tween-20 followed by incubation with anti-rabbit IgG-HRP (1:5000 dil in 4% BSA in PBS/0.5% Tween-20). The membrane was again washed three times and developed using ECL reagents (Amersham, Piscataway, N.J.).

In vitro Notch Cleavage ELISA. Notch cleavage was also assessed using an ELISA technique. Prior to setting up a reaction, the required number of wells in a 96-well half-area plate (Costar) were coated with 50 μl of Val-1744 antibody diluted 1:200 in 0.1 M NaHCO₃, pH 8.2. Plates were incubated overnight at 4° C. The Notch cleavage reaction was set up as follows. NusA-Notch (0.9 μM) was incubated with 70 mg/ml of solubilized γ-secretase in 50 mM Pipes, pH 7.0, 0.25% CHAPSO in a total volume of 25 μl overnight at 37° C. The next day, the plate coated with Val-1744 antibody was washed 3 times with PBS/0.05% Tween-20 and blocked using 4% BSA in PBS/0.05% Tween-20 for 1 hour. The cleavage reaction mix was diluted 14-fold using 4% BSA in PBS/0.05% Tween-20 and 50 μl was plated in triplicate and incubated for 3-4 hours at room temp. Plates were washed three times with PBS/0.05% Tween-20 and 50 μl anti-FLAG-HRP antibody (Sigma, St. Louis, Mo) used at 1:60000 dilution in 4% BSA/PBS/0.5% Tween-20 was added. This antibody was incubated for 45 min and the plate washed three times with PBS/0.5% Tween-20. TMB reagent (Kirkegaard & Perry) was mixed 1:1 and 50 μl was added to the wells. The color was allowed to develop for 1 hour and 50 l of 1M H₃PO₄ was added and the plates read at 450 nm on a SpectraMax Plus plate reader. When varying the Notch substrate concentration, 0.11 to 3.6 μM of NusA-Notch was used. When the enzyme concentration was varied, 2.1 to 68 μg/ml solubilized γ-secretase was used.

Inhibition of Notch Cleavage. Inhibitors (1 μl) were added to the cleavage reaction as 25× concentrations in 50% DMSO prior to the addition of the enzyme. Blanks and no inhibitor controls were adjusted to contain the same final DMSO concentration. IC₅₀′s were calculated for inhibitors using the 4-parameter logistic model in the GraFit 4.0 program.

### Example 2

### Results and Discussion

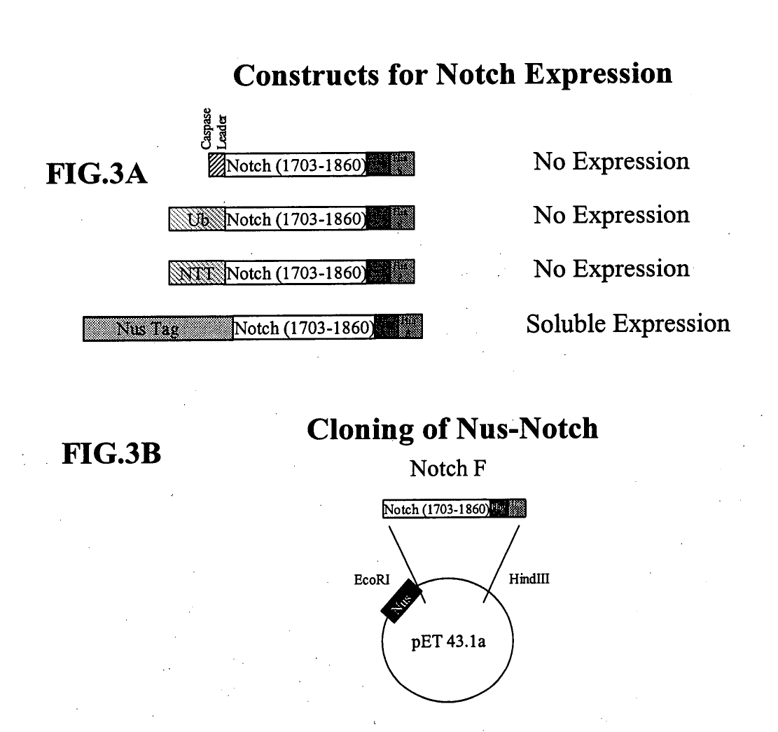

In order to produce a soluble Notch substrate, the inventors prepared a number of constructs in E. coli (FIG. 3a). These include the use of caspase leader, ubiquitin, N-terminal tau, and the Nus tag. Soluble expression was observed only with Nus fusion (Wilkinson et al., Bio/Technology 9:443-448, 1991). FIG. 3b shows cloning details for this construct. To facilitate purification and assay development, a His and a Flag tag were engineered to the C-terminus of Notch.

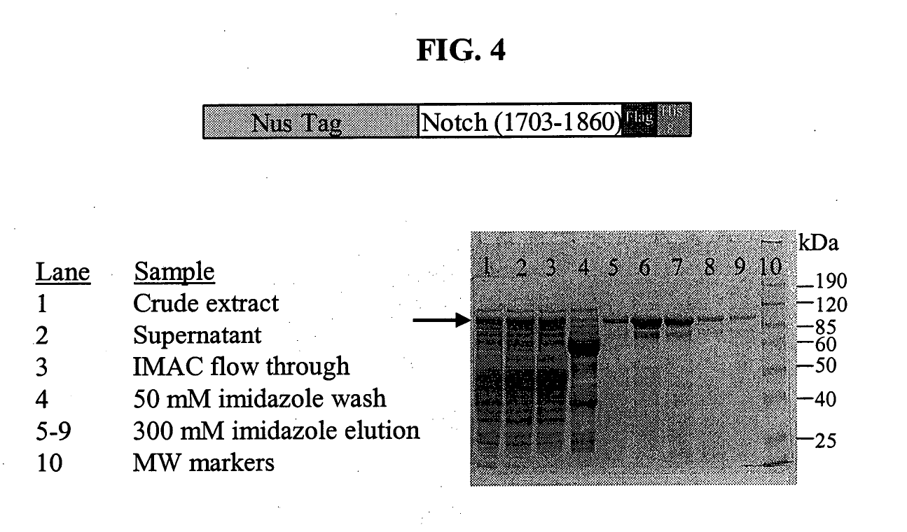

When the Nus-tagged Notch fusion protein was expressed in E. coli, a high level expression of the fusion protein corresponding to a 90 kDa band was observed on a SDS-PAGE (FIG. 4, Lane 1), a size expected from the fusion protein. When the total lysate was centrifuged, the fusion protein remained in the soluble fraction (FIG. 4, lane 2). Notch-containing fusion protein was purified from the soluble fraction by IMAC using nickel as the immobilized metal ion. FIG. 4 (lanes 5-9) shows various fractions eluted from the IMAC column by 300 mM imidazole. These fractions were pooled, dialyzed, and then used as a source of the substrate for γ-secretase cleavage.

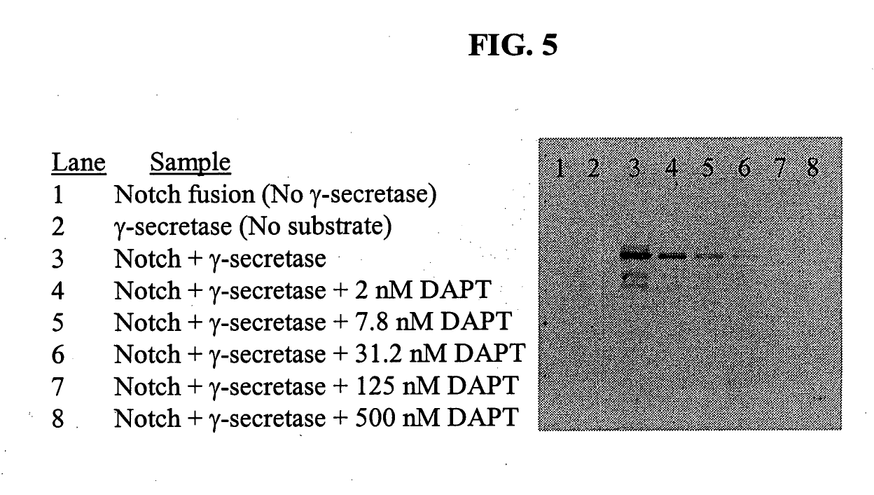

The technique for following a specific cleavage in the Notch protein is based on the specificity of Val-1744 antibody (Cell Signaling Technology). It is specific for cleaved Notch and does not cross react with uncleaved Notch protein. As shown in FIG. 5 (lane 3), the cleaved Notch protein was detected on a Western blot with antibody that is specific for Val-1744. As shown in lane 1, no cross reactivity to the uncleaved Notch fusion protein was observed. Moreover, this specific cleavage in Notch protein was inhibited in a dose-dependent manner by DAPT (Dovey et al., J. Neurochem. 76:173-181, 2001), a well-known potent inhibitor of γ-secretase (lanes 4-8, FIG. 5).

In order to determine if there is an additional cleavage in the Notch protein, the cleavage reaction was also monitored by a Western blot using the C-terminal Flag antibody. Out of the three immunoreactive bands, only one cleavage product was inhibitable by DAPT. Taken together, these results suggest that in vitro, γ-secretase-mediated cleavage seems to result in a specific cleavage at the 1743/1744, consistent with cell based studies showing the production of NICD from Notch (Kopan et al., Proc. Natl. Acad. Sci. 93:1683-1688, 1996; Schroeter et al., Nature, 393:382-386, 1998).

The above results showed that Notch fusion protein is susceptible to a γ-secretase-mediated specific cleavage which can be detected on a Western blot by a highly specific Val-1744 monoclonal antibody. Since Western blots are not quantitative, it is difficult to evaluate compounds for Notch inhibition and compare the inhibitory potencies to the Aβ ELISA.

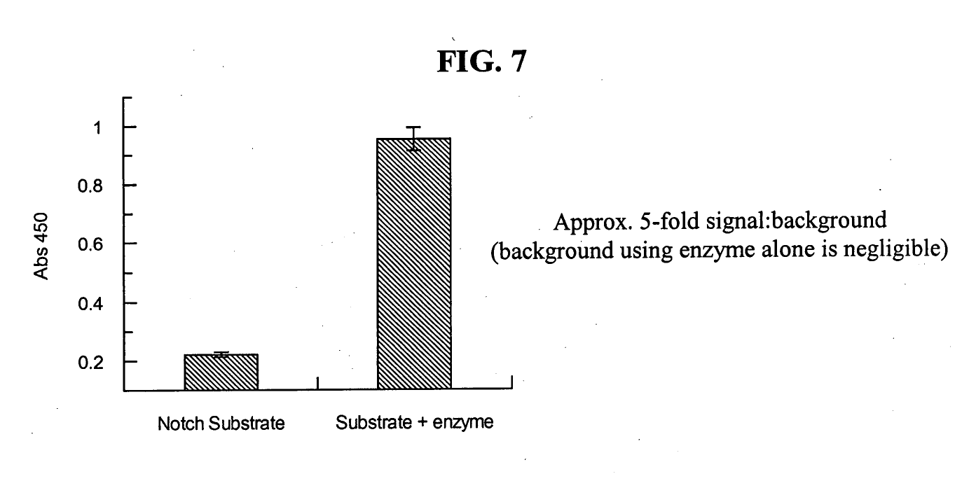

FIG. 6 shows a strategy for the development of a quantitative ELISA for Notch cleavage for comparison of inhibitory potency of compounds with the CT-100-based ELISA. It is based on a sandwich ELISA using the highly specific anti-Val 1744 and anti Flag antibodies to capture the N- and C-terminus of the NICD fragment from the Notch fusion protein in the presence of γ-secretase. This assay is performed as described in Example 1 under the heading “In vitro Notch Cleavage ELISA”. Essentially, in this sandwich ELISA, the microtiter plate is coated Val-1744 antibody. Meanwhile the NusA-Notch cleavage reaction is performed in which NusA-Notch is incubated with γ-secretase. The plate coated with Val-1744 antibody is washed with buffer and blocked with BSA. The cleavage reaction mixture is then added to the microtiter plate and incubated for an appropriate time period at room temp. Plates are washed anti-FLAG-HRP antibody (Sigma, St. Louis, Mo.) is added and again incubated for an appropriate time. The chromogenic substrate for HRP, TMB reagent (Kirkegaard & Perry) is added and the color once developed is read at 450 nm on a SpectraMax Plus plate reader. FIG. 7 shows cleavage of NusA-Notch fusion protein as detected by ELISA. A 5-fold signal to noise ratio was observed in the ELISA.

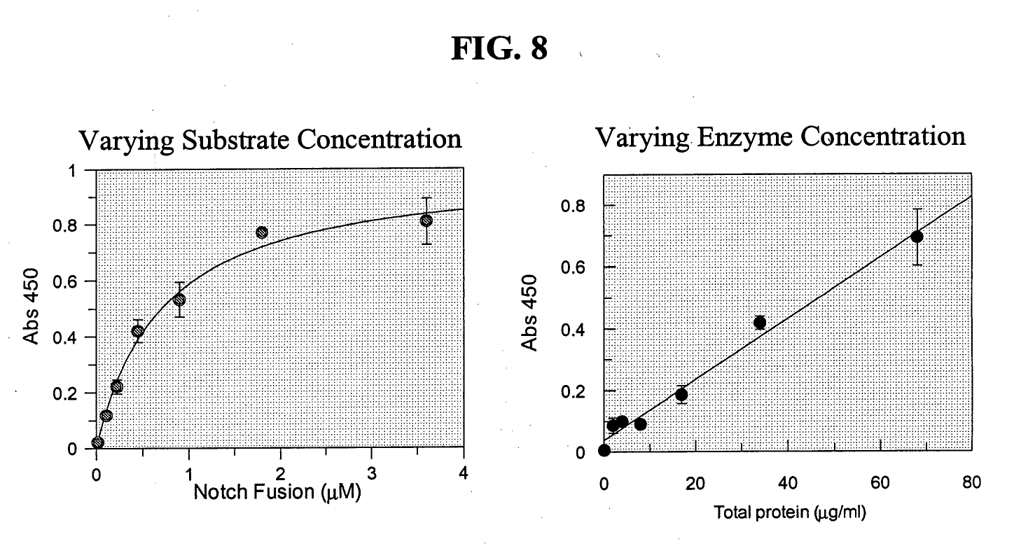

The enzymatic activity of solubilized γ-secretase for Notch cleavage was further characterized in the ELISA under defined experimental conditions. FIG. 8A shows the substrate dependence on product formation. The reaction obeys a Michaelis-Menten kinetics and the apparent Kₘ for hydrolysis of Notch fusion protein substrate by solubilized γ-secretase activity is about 0.7 μM under defined conditions. The observed Notch cleavage activity was also linear with protein concentration of solubilized membrane preparation containing γ-secretase activity (FIG. 8B).

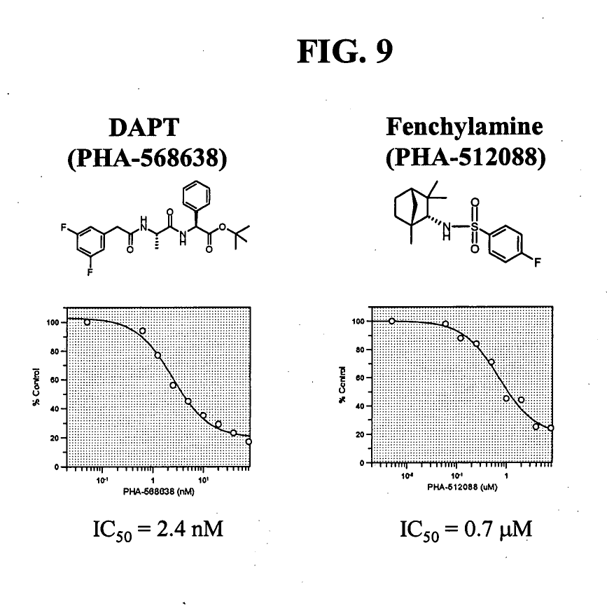

Further confirmation of γ-secretase-mediated Notch cleavage in the ELISA was obtained by inhibition studies using specific inhibitors of this enzyme reported in the literature. A potent inhibitor of γ-secretase, called DAPT, has been reported (Dovey et al., J. Neurochem. 76:173-181, 2001). As show in FIG. 9, DAPT (PHA-568638) inhibits Notch cleavage in a dose-dependent manner with an IC₅₀=2.4 nM. On the other hand, fenchylamine sulfonamide (PHA-512088) has been reported (Rishton et al., J. Med. Chem. 43:2297-2299, 2000) to inhibit γ-secretase activity in the low μM range in the CT-100 in vitro assay. As shown in FIG. 9, PHA-512088 inhibited r secretase-mediated Notch cleavage with an IC₅₀ of 0.7 μM in the ELISA under defined conditions. As shown, inhibitors (PHA-568638; PHA-512088) in the low nM to low gM range work very well in the assay. Taken together, these results show that in vitro enzymatic activity producing a NICD fragment from a Notch fusion protein substrate is due to γ-secretase.

Recently, cell based assays to follow APP and Notch cleavage in parallel have been reported (Karlstrom et al., J. Biol. Chem. 277:6763-6766, 2002). However, the present invention for the first time demonstrates an in vitro quantitative ELISA for following specific Notch cleavage (G1743-V1744) by γ-secretase. The present invention for the first time demonstrates that γ-secretase activity from HeLa cells specifically cleaves the Notch protein at the 1743-1744 junction.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

We claim:

1. A soluble fusion protein comprising recombinant Notch protein fused to the C-terminus of a NusA protein sequence.

2. The soluble fusion protein of claim 1, wherein said recombinant Notch protein comprises the S3 cleavage site of Notch.

3. The soluble fusion protein of claim 1, wherein said recombinant Notch protein is a vertebrate Notch protein.

4. The soluble fusion protein of claim 1, wherein said recombinant Notch protein is an invertebrate Notch protein.

5. The soluble fusion protein of claim 1, wherein said recombinant Notch protein is derived from mouse Notch protein having the sequence of SEQ ID NO:5.

6. The soluble fusion protein of claim 1, wherein said recombinant Notch protein comprises amino acids 1703 through 1860 of mouse Notch protein.

7. The soluble fusion protein of claim 1, further comprising a C-terminal His-tag.

8. The soluble fusion protein of claim 1, further comprising a C-terminal Flag-tag.

9. A polynucleotide comprising a nucleotide sequence that encodes a fusion protein according to claim 1.

10. A polynucleotide sequence that encodes a fusion protein of claim 1, wherein said polynucleotide sequence comprises a sequence set forth in SEQ ID NO:1.

11. An expression vector comprising a polynucleotide of claim 9.

12. The expression vector of claim 11, wherein said polynucleotide is operably linked to a promoter to promote expression of the protein encoded by the polynucleotide in a host cell.

13. A recombinant host cell transformed or transfected with a polynucleotide of claim 9.

14. A recombinant host cell transformed or transfected with an expression vector of claim 11.

15. A method of producing a solubilized Notch protein, said method comprising preparing a fusion protein wherein the said Notch protein is fused to the C-terminus of a NusA protein.

16. The method of claim 15, wherein said method comprises a recombinant production of said fusion protein, said method comprising:
a. preparing an expression construct comprising a nucleic acid that encodes a fusion protein comprising a Notch protein containing the amino acids of the S3 cleavage site of Notch linked at the C-terminus of a NusA protein;
b. transforming a host cell with said expression construct under conditions that facilitate the expression of said fusion protein; and
c. growing said transformed host cell in culture.

17. The method of claim 16, further comprising isolating said fusion protein from said transformed host in culture.

18. The method of claim 15, wherein said method comprises producing said fusion protein through chemical protein synthesis.

19. The method of claim 16, wherein said Notch protein comprises amino acids 1703 through 1860 of mouse Notch protein.

20. An in vitro method of assaying for γ-secretase mediated ε cleavage (1743/1744) of Notch protein comprising:
a. contacting a first composition comprising a mammalian γ-secretase complex or biologically active fragment thereof, with a second compositions comprising a fusion protein according to claim 1; and
b. measuring cleavage of the fusion protein.

21. An in vitro method of screening for modulators of γ-secretase mediated ε cleavage (1743/1744) of Notch protein, comprising the steps of:
(a) contacting a first composition comprising a mammalian γ-secretase complex or biologically active fragment thereof, with a second compositions comprising a fusion protein according to claim 1 in the presence and in the absence of a putative modulator compound; and
(b) measuring cleavage of the fusion protein in the presence and in the absence of a putative modulator compound; and
(c) identifying modulators which modulate the γ-secretase mediated cleavage of said fusion protein;
wherein a putative modulator compound produces a difference in γ-secretase cleavage in step (b).

22. The method of claim 20, wherein the γ-secretase complex of the first composition comprises a membrane fraction purified and isolated from mammalian cells or cells transformed or transfected with expression constructs comprising nucleotide sequences that encode the γ-secretase complex.

23. The method of claim 20, wherein said fusion protein is a solubilized Notch protein prepared according to any one of claims 15 through 19.

24. The method of claim 21, wherein the putative modulator compound modulates the γ-secretase cleavage of APP.

25. The method of claim 21, wherein the putative modulator compound inhibits the γ-secretase cleavage of APP to a greater extent than γ-secretase cleavage of Notch protein.

26. A method of producing a substrate for a γ-secretase assay comprising growing a host cell of claim 13 in a manner allowing expression of said fusion protein.

27. The method of claim 26, further comprising purifying said polypeptide.

28. The method of claim 26, wherein said host cell is selected from the group consisting of a mammalian host cell, a bacterial host cell and a yeast host cell.

29. The method of claim 28, wherein the cell is a Hela cell, a human embryonic kidney cell line 293 cell, a fibroblast, or a CHO cell.

30. A method of producing a substrate for a γ-secretase assay comprising growing a host cell of claim 14 in a manner allowing expression of said polypeptide.

31. The method of claim 30, further comprising purifying said polypeptide.

32. The method of claim 31, wherein said host cell is selected from the group consisting of a mammalian host cell, a bacterial host cell and a yeast host cell.

33. The method of claim 32, wherein the cell is a Hela cell, a human embryonic kidney cell line 293 cell, a human embryonic kidney cell line 293 cell, a fibroblast, or a CHO cell.

34. A kit for performing a γ-secretase assay comprising a γ-secretase substrate comprising a fusion protein according to claim 1.

35. The kit of claim 34, further comprising reagents for detecting the cleavage of said fusion protein.

36. A fusion protein comprising a NusA polypeptide fused to a Notch polypeptide comprising between 90 to 95% sequence identity with a NusA sequence of SEQ ID NO:14, wherein the Notch polypeptide comprises the transmembrane domain of Notch, and wherein the fusion protein is soluble in an aqueous solution.

37. A method for screening for a selective inhibitor of γ-secretase processing of amyloid precursor protein (APP), comprising:
a) providing a test compound which inhibits γ-secretase mediated cleavage of a polypeptide comprising an APP gamma secretase site; and
b) measuring gamma secretase cleavage of a fusion protein according to claim 1 in the presence and absence of the test compound;
wherein a test compound that preferentially inhibits gamma secretase cleavage of said polypeptide compared to cleavage of said fusion protein is a selective inhibitor of gamma secretase processing of APP.

38. A selective inhibitor identified by the method of claim 37.

39. A method of modulating γ-secretase activity in-vivo comprising a step of administering a selective inhibitor of claim 37 to a mammal in an effective amount to modulate γ-secretase activity in cells of said mammal.

40. A pharmaceutical composition comprising a selective inhibitor of claim 38 and a pharmaceutically acceptable carrier.

41. A method of treating a disease or condition characterized by an abnormal γ-secretase activity comprising administering to a subject in need of treatment a pharmaceutical composition of claim 40.

42. A use of a selective inhibitor identified according to the method of claim 37 in the manufacture of a medicament for the treatment of Alzheimer's disease.


SEQUENCE LISTING (14 sequences)

SEQ ID NO: 1 - DNA, 2190 bp, Artificial sequence
  DNA encoding synthetic fusion of notch and nus
atgaacaaagaaattttggctgtagttgaagccgtatccaatgaaaaggcgctacctcgcgagaagattttcgaagcattggaaagcgcgctggcgacagcaacaaagaaaaaatatgaacaagagatcgacgtccgcgtacagatcgatcgcaaaagcggtgattttgacactttccgtcgctggttagttgttgatgaagtcacccagccgaccaaggaaatcacccttgaagccgcacgttatgaagatgaaagcctgaacctgggcgattacgttgaagatcagattgagtctgttacctttgaccgtatcactacccagacggcaaaacaggttatcgtgcagaaagtgcgtgaagccgaacgtgcgatggtggttgatcagttccgtgaacacgaaggtgaaatcatcaccggcgtggtgaaaaaagtaaaccgcgacaacatctctctggatctgggcaacaacgctgaagccgtgatcctgcgcgaagatatgctgccgcgtgaaaacttccgccctggcgaccgcgttcgtggcgtgctctattccgttcgcccggaagcgcgtggcgcgcaactgttcgtcactcgttccaagccggaaatgctgatcgaactgttccgtattgaagtgccagaaatcggcgaagaagtgattgaaattaaagcagcggctcgcgatccgggttctcgtgcgaaaatcgcggtgaaaaccaacgataaacgtatcgatccggtaggtgcttgcgtaggtatgcgtggcgcgcgtgttcaggcggtgtctactgaactgggtggcgagcgtatcgatatcgtcctgtgggatgataacccggcgcagttcgtgattaacgcaatggcaccggcagacgttgcttctatcgtggtggatgaagataaacacaccatggacatcgccgttgaagccggtaatctggcgcaggcgattggccgtaacggtcagaacgtgcgtctggcttcgcaactgagcggttgggaactcaacgtgatgaccgttgacgacctgcaagctaagcatcaggcggaagcgcacgcagcgatcgacaccttcaccaaatatctcgacatcgacgaagacttcgcgactgttctggtagaagaaggcttctcgacgctggaagaattggcctatgtgccgatgaaagagctgttggaaatcgaaggccttgatgagccgaccgttgaagcactgcgcgagcgtgctaaaaatgcactggccaccattgcacaggcccaggaagaaagcctcggtgataacaaaccggctgacgatctgctgaaccttgaaggggtagatcgtgatttggcattcaaactggccgcccgtggcgtttgtacgctggaagatctcgccgaacagggcattgatgatctggctgatatcgaagggttgaccgacgaaaaagccggagcactgattatggctgcccgtaatatttgctggttcggtgacgaagcgactagtggttctggtcatcaccatcaccatcactccgcgggtaaagaaaccgctgctgcgaaatttgaacgccagcacatggactcgccaccgccaactggtctggtcccccggggcagcgcgggttctggtacgattgatgacgacgacaagagtccgggagctcgtggatccgaattcaatattccttacaagattgaggccgtgaagagtgagccggtggagcctccgctgccctcgcagctgcacctcatgtacgtggcagcggccgccttcgtgctcctgttctttgtgggctgtggggtgctgctgtcccgcaagcgccggcggcagcatggccagctctggttccctgagggtttcaaagtgtcagaggccagcaagaagaagcggagagagcccctcggcgaggactcagtcggcctcaagcccctgaagaatgcctcagatggtgctctgatggacgacaatcagaacgagtggggagacgaagacctggagaccaagaagttccggtttgaggagccagtagttctccctgacctgagtgatcagactgaccacagacagtggacccagcagcacctggacgctgctgacctgcgcatgtctgccatggccccaacaccgcctcagggggaggtggatgctgacgattataaagacgatgacgataaacaccatcaccatcaccatcaccattga

SEQ ID NO: 2 - protein, 729 aa, Artificial sequence
  Synthetic fusion protein sequence of notch and nus
MNKEILAVVEAVSNEKALPREKIFEALESALATATKKKYEQEIDVRVQIDRKSGDFDTFRRWLVVDEVTQPTKEITLEAARYEDESLNLGDYVEDQIESVTFDRITTQTAKQVIVQKVREAERAMVVDQFREHEGEIITGVVKKVNRDNISLDLGNNAEAVILREDMLPRENFRPGDRVRGVLYSVRPEARGAQLFVTRSKPEMLIELFRIEVPEIGEEVIEIKAAARDPGSRAKIAVKTNDKRIDPVGACVGMRGARVQAVSTELGGERIDIVLWDDNPAQFVINAMAPADVASIVVDEDKHTMDIAVEAGNLAQAIGRNGQNVRLASQLSGWELNVMTVDDLQAKHQAEAHAAIDTFTKYLDIDEDFATVLVEEGFSTLEELAYVPMKELLEIEGLDEPTVEALRERAKNALATIAQAQEESLGDNKPADDLLNLEGVDRDLAFKLAARGVCTLEDLAEQGIDDLADIEGLTDEKAGALIMAARNICWFGDEATSGSGHHHHHHSAGKETAAAKFERQHMDSPPPTGLVPRGSAGSGTIDDDDKSPGARGSEFNIPYKIEAVKSEPVEPPLPSQLHLMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLSDQTDHRQWTQQHLDAADLRMSAMAPTPPQGEVDADDYKDDDDKHHHHHHHH

SEQ ID NO: 3 - DNA, 525 bp, Artificial sequence
  Wildtype notch DNA sequence
aatattccttacaagattgaggccgtgaagagtgagccggtggagcctccgctgccctcgcagctgcacctcatgtacgtggcagcggccgccttcgtgctcctgttctttgtgggctgtggggtgctgctgtcccgcaagcgccggcggcagcatggccagctctggttccctgagggtttcaaagtgtcagaggccagcaagaagaagcggagagagcccctcggcgaggactcagtcggcctcaagcccctgaagaatgcctcagatggtgctctgatggacgacaatcagaacgagtggggagacgaagacctggagaccaagaagttccggtttgaggagccagtagttctccctgacctgagtgatcagactgaccacagacagtggacccagcagcacctggacgctgctgacctgcgcatgtctgccatggccccaacaccgcctcagggggaggtggatgctgacgattataaagacgatgacgataaacaccatcaccatcaccatcaccattga

SEQ ID NO: 4 - protein, 174 aa, Artificial sequence
  Wildtype notch protein sequence
NIPYKIEAVKSEPVEPPLPSQLHLMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLSDQTDHRQWTQQHLDAADLRMSAMAPTPPQGEVDADDYKDDDDKHHHHHHHH

SEQ ID NO: 5 - protein, 2531 aa, Mus musculus
MPRLLTPLLCLTLLPARAARGLRCSQPSGTCLNGGRCEVASGTEACVASGSFVGQRCQDPNPCLSTRCKNAGTCYVVDHGGIVDYACSCPLGFSGPLCLTPLDKPCLANPCRNGGTCDLLTLTEYKCRCSPGWSGKSCQQADPCASNPCANGGQCLPFESSYICRCPPGFHGPTCRQDVNECSQNPGLCRHGGHCHNEIGSYRCACCATHTGPHCELPYVPCSPSPCQNGATCRPTGDTTHECACLPGFAGQNCEENVDDCPGNNCKNGGACVDGVNTYNCRCPPEVTGQYCTEDVDECQLMPNACQNAGTCHNTHGGYNCVCVNGWTGEDCSENIDDCASAACFQGATCHDRVASFYCECPHGRTGLLCHLKHACISNPCNEGSNCDTNPVNGKRICTCPSGYTGPACSQDVDECDLGANRCEHAGKCLNTLGSFECQCLQGYTGPGCEIDVNECISNPCQNDATCLDQIGEFQCICMPGYEGVYCEINTDECASSPCLHNGHCMDKIHEFQCQCPKGFNGHLCQYDVDECASTPCKNGAKCLDGPNTYTCVCTEGYTGTHCEVDIDECDPDPCHYGSCKDGVATFTCLCQPGYTGHHCETNINECHSQPCRHGGTCQDRDNSYLCLCLKGTTGPNCEINLDDCASNPCDSGTCLDKIDGYECACEPGYTGSMCNVNIDECAGSPCHNGGTCEDGIAGFTCRCPEGYHDPTCLSEVNECNSNPCIHGACRDGLNGYKCDCAPGWSGTNCDINNNECESNPCVNGGTCKDMTSGYVCTCREGFSGPNCQTNINECASNPCLNQGTCIDDVAGYKCNCPLPYTGATCEVVLAPCATSPCKNSGVCKESEDYESFSCVCPTGWQGQTCEVDINECVKSPCRHGASCQNTNGSYRCLCQAGYTGRNCESDIDDCRPNPCHNGGSCTDGINTAFCDCLPGFQGAFCEEDINECASNPCQNGANCTDCVDSYTCTCPVGFNGIHCENNTPDCTESSCFNGGTCVDGINSFTCLCPPGFTGSYCQYDVNECDSRPCLHGGTCQDSYGTYKCTCPQGYTGLNCQNLVRWCDSAPCKNGGRCWQTNTQYHCECRSGWTGVNCDVLSVSCEVAAQKRGIDVTLLCQHGGLCVDEGDKHYCHCQAGYTGSYCEDEVDECSPNPCQNGATCTDYLGGFSCKCVAGYHGSNCSEEINECLSQPCQNGGTCIDLTNSYKCSCPRGTQGVHCEINVDDCHPPLDPASRSPKCFNNGTCVDQVGGYTCTCPPGFVGERCEGDVNECLSNPCDPRGTQNCVQRVNDFHCECRAGHTGRRCESVINGCRGKPCKNGGVCAVASNTARGFICRCPAGFEGATCENDARTCGSLRCLNGGTCISGPRSPTCLCLGSFTGPECQFPASSPCVGSNPCYNQGTCEPTSENPFYRCLCPAKFNGLLCHILDYSFTGGAGPDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLSDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDRRKRLEASADANIQDNMGRTPLHAAVSADAQGVFQILLRNRATDLDARMHDGTTPLILAARLAVEGMLEDLINSHADVNAVDDLGKSALHWAAAVNNVDAAVVLLKNGANKDIENNKEETSLFLSIRRESYETAKVLLDHFANRDITDHMDRLPRDIAQERMHHDIVRLLDEYNLVRSPQLHGTALGGTPTLSPTLCSPNGYPGNLKSATQGKKARKPSTKGLACGSKEAKDLKARRKSSQDGKGWLLDSSSSMLSPVDSLESPHGYLSDVASHPLLPSPFQQSPSMPLSHLPGMPDTHLGISHLNVAAKPEMAALAGGSRLAFEHPPPRLSHLPVASSACTVLSTNGTGAMNFTVGAPASLNGQCEWLPRLQNGMVPSQYNPLRPGVTPGTLSTQAAGLQHSMMGPLHSSLSTNTLSPIIYQGLPNTRLATQPHLVQTQQVQPQNLPLQPQNLQPPSQPHLSVSSAANGHLGRSFLSGEPSQADVQPLGPSSLPVHTILPQESQALPTSLPSSMVPPMTTTQFLTPPSQHSYSSSPVDNTPSHQLQVPEPTFLTPSPESPDQWSSSSPHSNISDWSEGISSPPTTMPSQITHIPEAFK

SEQ ID NO: 6 - protein, 2444 aa, Homo sapiens
  misc_feature (891)..(891): Xaa = Any or unknown amino acid
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPRLCRHGGTCHNEVGSYRCVCRATHTGPNCERPYVPCSPSPCQNGGTCRPTGDVTHECACLPGFTGQNCEENIDDCPGNNCKNGGACVDGVNTYNCPCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCMCMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRLRGTCQDPDNAYLCFCLKGTTGPNCEINLDDCASSPCDSGTCLDKIDGYECACEPGYTGSMCNSNIDECAGNPCHNGGTCEDGINGFTCRCPEGYHDPTCLSEVNECNSNPCVHGACRDSLNGYKCDCDPGWSGTNCDINNNECESNPCVNGGTCKDMTSGIVCTCREGFSGPNCQTNINECASNPCLNKGTCIDDVAGYKCNCLLPYTGATCEVVLAPCAPSPCRNGGECRQSEDYESFSCVCPTAGAKGQTCEVDINECVLSPCRHGASCQNTHGXYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHVVNECDSRPCLLGGTCQDGRGLHRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRXQHGQLWFPEGFKVSEASKKKRREXLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADV

SEQ ID NO: 7 - protein, 30 aa, Artificial sequence
  Amino acid sequence surrounding the transmembrane domains of APP
SNKGAIIGLMVGGVVIATVIVITLVMLKKK

SEQ ID NO: 8 - protein, 30 aa, Artificial sequence
  Sequence surrounding the transmembrane domains of E-cathedrin
IPEWLIILASLLLALALILAVCIAVNSRRR

SEQ ID NO: 9 - protein, 30 aa, Artificial sequence
  Sequence surrounding the transmembrane domains of Notch-1
PSQLHLMYVAAAAFVLLFFVGCGVLLSRKR

SEQ ID NO: 10 - protein, 158 aa, Artificial sequence
  Sequence surrounding the transmembrane domains of Notch-1
NIPYKIEAVKSEPVEPPLPSQLHLMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLSDQTDHRQWTQQHLDAADLRMSAMAPTPPQGEVDAD

SEQ ID NO: 11 - protein, 8 aa, Artificial sequence
  C-terminal flag sequence
DYKDDDDK

SEQ ID NO: 12 - protein, 16 aa, Artificial sequence
  Flag/8 his tag
DYKDDDDKHHHHHHHH

SEQ ID NO: 13 - DNA, 1665 bp, Artificial sequence
  Nucleic acid encoding NusA
atgaacaaagaaattttggctgtagttgaagccgtatccaatgaaaaggcgctacctcgcgagaagattttcgaagcattggaaagcgcgctggcgacagcaacaaagaaaaaatatgaacaagagatcgacgtccgcgtacagatcgatcgcaaaagcggtgattttgacactttccgtcgctggttagttgttgatgaagtcacccagccgaccaaggaaatcacccttgaagccgcacgttatgaagatgaaagcctgaacctgggcgattacgttgaagatcagattgagtctgttacctttgaccgtatcactacccagacggcaaaacaggttatcgtgcagaaagtgcgtgaagccgaacgtgcgatggtggttgatcagttccgtgaacacgaaggtgaaatcatcaccggcgtggtgaaaaaagtaaaccgcgacaacatctctctggatctgggcaacaacgctgaagccgtgatcctgcgcgaagatatgctgccgcgtgaaaacttccgccctggcgaccgcgttcgtggcgtgctctattccgttcgcccggaagcgcgtggcgcgcaactgttcgtcactcgttccaagccggaaatgctgatcgaactgttccgtattgaagtgccagaaatcggcgaagaagtgattgaaattaaagcagcggctcgcgatccgggttctcgtgcgaaaatcgcggtgaaaaccaacgataaacgtatcgatccggtaggtgcttgcgtaggtatgcgtggcgcgcgtgttcaggcggtgtctactgaactgggtggcgagcgtatcgatatcgtcctgtgggatgataacccggcgcagttcgtgattaacgcaatggcaccggcagacgttgcttctatcgtggtggatgaagataaacacaccatggacatcgccgttgaagccggtaatctggcgcaggcgattggccgtaacggtcagaacgtgcgtctggcttcgcaactgagcggttgggaactcaacgtgatgaccgttgacgacctgcaagctaagcatcaggcggaagcgcacgcagcgatcgacaccttcaccaaatatctcgacatcgacgaagacttcgcgactgttctggtagaagaaggcttctcgacgctggaagaattggcctatgtgccgatgaaagagctgttggaaatcgaaggccttgatgagccgaccgttgaagcactgcgcgagcgtgctaaaaatgcactggccaccattgcacaggcccaggaagaaagcctcggtgataacaaaccggctgacgatctgctgaaccttgaaggggtagatcgtgatttggcattcaaactggccgcccgtggcgtttgtacgctggaagatctcgccgaacagggcattgatgatctggctgatatcgaagggttgaccgacgaaaaagccggagcactgattatggctgcccgtaatatttgctggttcggtgacgaagcgactagtggttctggtcatcaccatcaccatcactccgcgggtaaagaaaccgctgctgcgaaatttgaacgccagcacatggactcgccaccgccaactggtctggtcccccggggcagcgcgggttctggtacgattgatgacgacgacaagagtccgggagctcgtggatccgaattc

SEQ ID NO: 14 - protein, 555 aa, Artificial sequence
  Protein sequence encoding NusA
MNKEILAVVEAVSNEKALPREKIFEALESALATATKKKYEQEIDVRVQIDRKSGDFDTFRRWLVVDEVTQPTKEITLEAARYEDESLNLGDYVEDQIESVTFDRITTQTAKQVIVQKVREAERAMVVDQFREHEGEIITGVVKKVNRDNISLDLGNNAEAVILREDMLPRENFRPGDRVRGVLYSVRPEARGAQLFVTRSKPEMLIELFRIEVPEIGEEVIEIKAAARDPGSRAKIAVKTNDKRIDPVGACVGMRGARVQAVSTELGGERIDIVLWDDNPAQFVINAMAPADVASIVVDEDKHTMDIAVEAGNLAQAIGRNGQNVRLASQLSGWELNVMTVDDLQAKHQAEAHAAIDTFTKYLDIDEDFATVLVEEGFSTLEELAYVPMKELLEIEGLDEPTVEALRERAKNALATIAQAQEESLGDNKPADDLLNLEGVDRDLAFKLAARGVCTLEDLAEQGIDDLADIEGLTDEKAGALIMAARNICWFGDEATSGSGHHHHHHSAGKETAAAKFERQHMDSPPPTGLVPRGSAGSGTIDDDDKSPGARGSEF